(19) United States
(12) Patent Application Publication
(10) Pub. No.: US 2024/0303817 A1
(43) Pub. Date: Sep. 12, 2024

(54) IMAGE PROCESSING AND ARTIFICIAL INTELLIGENCE TECHNIQUES FOR ANNULOPLASTY RING DETERMINATIONS

(71) Applicant: EDWARDS LIFESCIENCES CORPORATION, Irvine, CA (US)

(72) Inventors: Stephen Epstein, Millersville, MD (US); Wesley Paul Nicholson, Sunderland, MD (US)

(21) Appl. No.: 18/657,646

(22) Filed: May 7, 2024

Related U.S. Application Data
(63) Continuation of application No. PCT/US2022/050066, filed on Nov. 16, 2022.
(60) Provisional application No. 63/264,567, filed on Nov. 24, 2021.

(51) Int. Cl. G06T 7/00; A61B 34/00; A61B 34/10; G06T 7/62; G06T 11/00; G06V 10/77; G16H 30/40

(52) U.S. Cl. CPC G06T 7/0016; A61B 34/10; A61B 34/25; G06T 7/62; G06T 11/00; G06V 10/7715; G16H 30/40; A61B 2034/104; A61B 2034/105; G06T 2200/24; G06T 2207/20081; G06T 2207/30048; G06T 2207/30052; G06T 2210/41; G06V 2201/031

(57) ABSTRACT

Techniques relate to analyzing image data to determine an annuloplasty ring to implant for an annuloplasty procedure. For example, image data can be received that depicts a heart valve. The image data can be analyzed to identify one or more image features that represent one or more anatomical features of the heart valve. Based on the one or more image features, heart data can be generated that indicates a measurement and/or another characteristic of the heart valve. An annuloplasty ring can be determined based on the heart valve data and/or annuloplasty ring data indicating characteristics of one or more annuloplasty rings. User interface data can then be generated that indicates the annuloplasty ring.

### CROSS REFERENCE TO RELATED APPLICATION

This application is a continuation of International Patent Applicant No. PCT/US2022/050066, filed Nov. 16, 2022, which claims the benefit of U.S. Patent Application No. 63/264,567, filed on Nov. 24, 2021, the entire disclosures all of which are incorporated by reference for all purposes.

### BACKGROUND

### Field

The present disclosure relates to the field of medical devices and procedures.

### Description of related art

An annuloplasty is a procedure to tighten or reinforce the ring (annulus) around a valve in the heart. For example, due to various factors, two or more leaflets of a heart valve may not coapt properly, resulting in regurgitation of the blood flow (e.g., backwards blood flow) and/or other issues. To address such situations, an annuloplasty ring may be attached (e.g., sewn) to the annulus of the heart valve to pull the leaflets together for proper coaptation and to re-establish proper valve function.

### SUMMARY

In examples, the present disclosure relates to a system comprising control circuitry and memory communicatively coupled to the control circuitry. The control circuitry stores executable instructions that, when executed by the control circuitry, cause the control circuitry to perform operations comprising receiving image data depicting a heart valve, identifying one or more image features in the image data that represent one or more anatomical features of the heart valve, and based at least in part on the one or more image features, generating heart valve data indicating a measurement of the heart valve. The operations further comprise obtaining annuloplasty ring data indicating one or more characteristics of an annuloplasty ring, based at least in part on the heart valve data and the annuloplasty ring data, identifying the annuloplasty ring for implantation on the heart valve, and generating user interface data indicating the annuloplasty ring.

In some instances, the operations further comprise receiving additional image data depicting another heart valve before a procedure, receiving post-operative data indicating an effectiveness of another annuloplasty ring implanted on the other heart valve, and based at least in part on the additional image data and the post-operative data, performing one or more machine-learning techniques to generate a machine-trained model. The identifying the annuloplasty ring can include using the machine-trained model.

In some instances, the operations further comprise causing the image data to be displayed and receiving user input data indicating the one or more image features in the image data. The identifying the one or more image features can be based at least in part on the user input data.

In some instances, the operations further comprise performing image processing on the image data. Further, the identifying the one or more image features can be based at least in part on the image processing.

In some instances, the user interface data indicates at least one of a size of the annuloplasty ring, a shape of the annuloplasty ring, whether the annuloplasty ring is a closed or open ring, or a flexibility of the annuloplasty ring.

In some instances, the image data depicts a fiduciary marker indicating a predetermined distance. The generating heart valve data can be based at least in part on the predetermined distance.

In some instances, the measurement of the heart valve includes at least one of a surface area of a leaflet of the heart valve, a height of the leaflet, a surface area defined by an annulus of the heart valve, a height of the annulus, or an inter-commissural distance of the heart valve.

In some instances, the one or more characteristics of the annuloplasty ring comprise at least one of a size of the annuloplasty ring, a shape of the annuloplasty ring, a type of suture feature of the annuloplasty ring, whether the annuloplasty ring is a closed or open ring, or a flexibility of the annuloplasty ring.

In examples, the present disclosure relates to a method comprising capturing image data using an imaging device, the image data depicting a heart valve, performing, by control circuitry, image processing on the image data to identify multiple image features that represent anatomical features of the heart valve, respectively, and generating, by the control circuitry, heart valve data indicative of a measurement associated with the multiple image features. Further, the method comprises retrieving, by the control circuitry, annuloplasty ring data indicative of one or more characteristics of an annuloplasty ring, determining, by the control circuitry and based at least in part on the heart valve data and the annuloplasty ring data, to use the annuloplasty ring for the heart valve, and generating, by the control circuitry, user interface data indicative of the annuloplasty ring.

In some instances, the method further comprises placing a fiduciary marker within the field-of-view of the imaging device. The fiduciary marker can indicate a distance. The image data can depict the fiduciary marker and the heart valve data can be generated is based at least in part on the distance indicated by the fiduciary marker.

In some instances, the method further comprises receiving additional image data depicting another heart valve before a procedure, receiving post-operative data indicating an effectiveness of another annuloplasty ring implanted on the other heart valve, and based at least in part on the additional image data and the post-operative data, performing one or more machine-learning techniques to generate a machine-trained model. The determining to use the annuloplasty ring for the heart valve can include using the machine-trained model. The post-operative data can include user input indicating the effectiveness of the other annuloplasty implanted on the other heart valve.

In some instances, the user interface data indicates a size of the annuloplasty ring.

In examples, the present disclosure relates to one or more non-transitory computer-readable media storing computer-executable instructions that, when executed by control circuitry, instruct the control circuitry to perform operations comprising receiving pre-implantation image data depicting a heart valve, identifying one or more features in the pre-implantation image data that represent one or more anatomical features of the heart valve, and based at least in part on the one or more features in the pre-implantation image data, generating heart valve data indicating a measurement of the heart valve. The operations further comprise based at least in part on the heart valve data, using a machine-trained model to determine an annuloplasty ring to implant on the heart valve, and generating recommendation data indicating the annuloplasty ring.

In some instances, the operations further comprise receiving additional pre-implantation image data depicting another heart valve before a procedure, receiving post-operative data indicating an effectiveness of another annuloplasty ring implanted on the other heart valve, and based at least in part on the additional pre-implantation image data and the post-operative data, training a model to create the machine-trained model.

In some instances, the operations further comprise causing the pre-implantation image data to be displayed and receiving user input data indicating the one or more features in the image data. The identifying the one or more image features in the pre-implantation image data can be based at least in part on the user input data.

In some instances, the operations further comprise performing image processing on the image data. The identifying the one or more image features in the pre-implantation image data can be based at least in part on the image processing.

In some instances, the recommendation data indicates a size of the annuloplasty ring.

In some instances, the measurement of the heart valve includes at least one of a surface area of a leaflet of the heart valve or a height of the leaflet. Further, in some instances, the measurement of the heart valve includes at least one of a surface area defined by an annulus of the heart valve, a height of the annulus, or a distance between commissures of the heart valve.

Each method disclosed herein also encompass one or more simulations of the method, which are useful, for example, for teaching, demonstration, testing, device development, and procedure development. For example, methods for treating or diagnosing a patient include corresponding simulated methods performed on simulated patients. Suitable simulated patients or anthropogenic ghosts can include any combination of physical and virtual elements. Examples of physical elements include whole human or animal cadavers, or any portion thereof, including, organ systems, individual organs, or tissue; and manufactured cadaver, organ system, organ, or tissue simulations. Examples of virtual elements include visual simulations, which can be displayed on a screen; projected on a screen, surface, space, or volume; and holographic images. The simulation can also include one or more of another type of sensory input, for example, auditory, tactile, and olfactory stimuli.

For purposes of summarizing the disclosure, certain aspects, advantages and features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the disclosure. In addition, various features of different disclosed examples can be combined to form additional examples, which are part of this disclosure. Throughout the drawings, reference numbers may be reused to indicate correspondence between reference elements.

FIG. 1 illustrates a cross-sectional view of an example heart.

FIG. 2 illustrates a top/surgeon's view of a mitral valve of the heart from FIG. 1.

FIGS. 3A and 3B illustrate top and cross-sectional views, respectively, of an example mitral valve where the annulus is dilated and deformed causing mitral regurgitation.

FIGS. 4A and 4B illustrate to views of example mitral valves with ruptured and elongated chordae, respectively, both causing mitral regurgitation.

FIGS. 5A and 5B illustrate top and cross-sectional views, respectively, of an example mitral valve with symptoms of Barlow's disease with excess tissue and irregularly thickened leaflets.

FIGS. 6A and 6B illustrate top and cross-sectional views, respectively, of an example mitral valve having fibro-elastic deficiency with thinned leaflets and with excess tissue.

FIGS. 7A and 7B illustrate top and cross-sectional views, respectively, of an example mitral valve in Marfan's disease with excess and thin tissue and elongated chordae.

FIG. 8 illustrates an example annuloplasty ring implanted on a mitral valve.

FIG. 9 illustrates an example architecture to implement one or more of the techniques discussed herein.

FIG. 10 illustrates example techniques to capture image data of a marker to assist in identifying one or more measurements/characteristics of the anatomy of a patient.

FIG. 11 illustrates example image processing techniques to identify one or more features of image data.

FIG. 12 illustrates an example user interface to provide information regarding a characteristic and/or measurement of an anatomical feature.

FIG. 13 illustrates example techniques to determine an annuloplasty ring for implantation.

FIG. 14 illustrates examples techniques to train a model to determine an annuloplasty ring.

FIGS. 15A illustrates a top view of an example open/partial annuloplasty ring.

FIG. 15B illustrates a top view of the example annuloplasty ring of FIG. 15A implanted on a mitral valve.

FIGS. 16A illustrates a top view of another example open/partial annuloplasty ring.

FIG. 16B illustrates a top view of the example annuloplasty ring of FIG. 16A implanted on a mitral valve.

FIG. 17A illustrates a side view of an example closed annuloplasty ring.

FIG. 17B illustrates a top view of the annuloplasty ring of FIG. 17A.

FIG. 17C illustrates a top view of the annuloplasty ring of FIG. 17A implanted on a mitral valve.

FIG. 18A illustrates a side view of another example closed annuloplasty ring.

FIG. 18B illustrates a top view of the annuloplasty ring of FIG. 18A.

FIG. 18C illustrates a top view of the annuloplasty ring of FIG. 18A implanted on a mitral valve.

FIG. 19A illustrates a side view of an example closed annuloplasty ring having an asymmetrical form.

FIG. 19B illustrates a top view of the annuloplasty ring of FIG. 19A.

FIG. 19C illustrates a top view of the annuloplasty ring of FIG. 19A implanted on a mitral valve.

FIG. 20 illustrates an example flow diagram of a process to provide a recommendation regarding an annuloplasty ring to implant on a heart valve.

FIG. 21 illustrates an example flow diagram of a process to train a model to determine an annuloplasty ring to recommend for a situation.

### DETAILED DESCRIPTION

The headings provided herein are for convenience only and do not necessarily affect the scope or meaning of the claimed subject matter.

Although certain examples are disclosed below, the subject matter extends beyond the specifically disclosed examples to other alternative examples and/or uses, and to modifications and equivalents thereof. Thus, the scope of the claims that may arise here from is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for instance, various examples may be carried out in a manner that achieves or optimizes one advantage or group of advantages as taught herein without necessarily achieving other aspects or advantages as may also be taught or suggested herein.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

### Overview

As noted above, an annuloplasty procedure can be performed to remodel or reinforce the ring (annulus) around a valve in the heart. Such procedure involves attaching a structure (e.g., annuloplasty ring) to the annulus of the heart valve. Various types of annuloplasty rings have been developed to satisfy the myriad of contexts in which an annuloplasty ring may be implanted (e.g., different sized heart valves, heart valve abnormalities/issues, physician preferences, etc.). For instance, annuloplasty rings come in different sizes, shapes, materials, suture features for attachment, and so on, which provide physicians with options for an annuloplasty procedure. In many cases, a physician can use one or more ring sizers to determine a size of an annuloplasty ring to use. The physician can overlay D-shaped plates (e.g., the ring sizers) of different sizes onto the heart valve to identify an optimal size of an annuloplasty ring for the specific heart valve. However, in some cases it may be difficult to select the appropriate ring size or type of ring, due to the complicated structure of the heart valve. For example, since a leaflet can take various shapes, sizes, etc., it may be challenging for a physician to align features of a ring sizer with the features of the heart valve. Further, the size of the heart valve can be in between two ring sizers, requiring the physician to select a larger or smaller annuloplasty ring without necessarily knowing which ring size is most appropriate. Moreover, many ring sizers generally only account for a single or limited number of parameters, a distance between two points. As such, it may often be difficult to select an annuloplasty ring that has the appropriate size, shape, material, suture features, etc. In some cases after implanting an annuloplasty ring, the heart valve can continue to exhibit undesirable characteristics, such as regurgitation (in the case of using too large of an annuloplasty ring), excessive tissue that folds into the valve (in the case of implanting too small of an annuloplasty ring), and so on, which may ultimately require an additional surgery to replace the initial annuloplasty ring and/or lead to an ineffective procedure.

This disclosure describes techniques related to obtaining data regarding a heart valve and processing such data to determine an annuloplasty ring to implant at the heart valve. In some examples, the techniques can receive/capture image data regarding a heart valve and process the image data to identify one or more features depicted in the image data that represent one or more anatomical features of the heart valve. The techniques can generate heart valve data indicating one or more measurements or other characteristics of the heart valve based on the one or more image features. The techniques can use the heart valve data to determine an annuloplasty ring that is most appropriate for the heart valve and provide output data indicative of the annuloplasty ring. For instance, a physician can view information regarding a type of ring (e.g., size, identifier, etc.) and, if desired, select the annuloplasty ring for the procedure. In some instances, a model can be trained using heart valve data from different patients and machine learning. The model can be implemented to determine an annuloplasty ring that is most appropriate for a particular situation. As such, the techniques discussed herein can assist in more accurately selecting an optimal annuloplasty ring for a particular context, in comparison to other solutions. In some examples, the techniques can perform image processing or other techniques to accurately identify one or more characteristics/measurements of a heart valve and use such information to recommend an annuloplasty ring. In some cases, multiple characteristics/measurements can be identified/extracted to formulate a recommendation, which can assist in more accurately selecting an annuloplasty ring, in comparison to other solutions which rely on limited information.

### Example Heart Anatomy

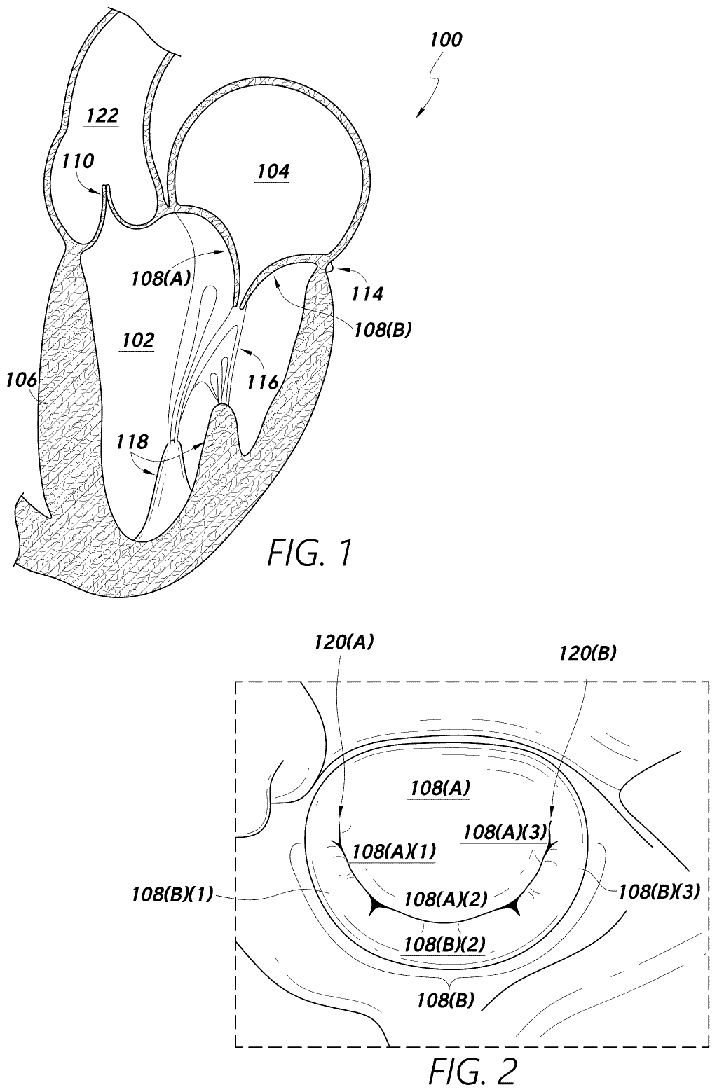

FIGS. 1 and 2 illustrates various features of an example healthy/normal heart 100. FIG. 1 illustrates a cross-sectional view of the heart 100, while FIG. 2 illustrates a top/surgeon's view looking at the mitral valve of the heart 100. The heart 100 includes four chambers, namely the left ventricle 102, the left atrium 104, the right ventricle (partially illustrated), and the right atrium (not illustrated). A wall of muscle, referred to as the septum, separates the left-side chambers from the right-side chambers. In particular, an atrial septum wall portion separates the left atrium 104 from the right atrium, whereas a ventricular septum wall portion 106 separates the left ventricle 102 from the right ventricle.

The heart 100 includes four valves for aiding the circulation of blood therein. Heart valves can generally comprise a relatively dense fibrous ring, referred to as the annulus, as well as a plurality of leaflets or cusps attached to the annulus. Generally, the size and position of the leaflets or cusps can be such that when the heart contracts, the resulting increased blood pressure produced within the corresponding heart chamber forces the leaflets at least partially open to allow flow from the heart chamber. As the pressure in the heart chamber subsides, the pressure in the subsequent chamber or blood vessel can become dominant and press back against the leaflets. As a result, the leaflets/cusps come in apposition to each other, thereby closing the flow passage.

The left ventricle 102 is the primary pumping chamber of the heart 100. A healthy left ventricle is generally conical or apical in shape in that it is longer (with respect to the mean electrical axis of the heart 100) than it is wide (with respect to a transverse axis extending between opposing walls of the left ventricle 102 at their widest point) and descends from a base with a decreasing cross-sectional diameter and/or circumference to the point or apex . Generally, the apical region of the heart 100 can be considered the bottom region of the heart 100 that is within the left and/or right ventricular region but is distal to the mitral valve 108 and tricuspid valve and disposed toward the tip of the heart 100.

The pumping of blood from the left ventricle 102 is accomplished by a squeezing motion and a twisting or torsional motion. The squeezing motion occurs between the lateral walls of the left ventricle 102 and the septum 106. The twisting motion is a result of contraction of heart muscle fibers that extend in a generally circular or spiral direction around the heart 100. When these fibers contract, they produce a gradient of angular displacements of the myocardium from the apex to the base about the mean electrical axis of the heart 100. The resultant force vectors extend at angles from about 30-60 degrees to the flow of blood through the aortic valve 110 and ascending aorta. The contraction of the heart 100 is manifested as a counterclockwise rotation of the apex relative to the base, when viewed from the apex (e.g., inferior view of the heart 100). The contractions of the heart 100, in connection with the filling volumes of the left atrium 104 and ventricle 102, respectively, can result in relatively high fluid pressures in the left side of the heart 100 at least during certain phase(s) of the cardiac cycle.

The primary roles of the chambers of the left side of the heart 100 (e.g., left atrium 104 and left ventricle 102) are to act as holding chambers for blood returning from the lungs (not shown) and to act as a pump to transport blood to other areas of the heart 100. The left atrium 104 receives oxygenated blood from the lungs via the pulmonary veins, which enters the left atrium 104 via the pulmonary vein ostia. The oxygenated blood that is collected from the pulmonary veins in the left atrium 104 enters the left ventricle 102 through the mitral valve 108. Deoxygenated blood enters the right atrium through the inferior and superior vena cava. The right side (e.g., right atrium and right ventricle) of the heart 100 then pumps this deoxygenated blood into the pulmonary arteries around the lungs. There, fresh oxygen enters the blood stream, and the blood moves to the left side of the heart 100 via the network of pulmonary veins that ultimately terminate at the left atrium 104.

The valves of the heart 100 include the mitral valve 108, which generally has two cusps/leaflets and separates the left atrium 104 from the left ventricle 102. The mitral valve 108 can generally be configured to open during diastole so that blood in the left atrium 104 can flow into the left ventricle 102, and close during systole to prevent blood from leaking back into the left atrium 104. The bases of the two valve leaflets are attached to a circular fibrous structure of the heart 100 called the annulus 114, and their free edges to chordae tendineae 116 arising from papillary muscles 118 of the left ventricle 102. An anterior leaflet 108(A) is relatively large and attaches to the anterior segment of the annulus 114, while a posterior leaflet 108(B) is smaller but extends further circumferentially and attaches to the posterior segment of the annulus 114, as shown in FIG. 2. The posterior leaflet 108(B) presents three scallops identified as 108(B)(1)-108(B)(3), while the corresponding non-scalloped parts of the anterior leaflet 108(A) are identified as 108(A)(1)-108(A)(3). The anterior leaflets 108(A) and the posterior leaflets 108(B) join and insert into the annulus 114 at the commissures 120, namely the anterior commissure 120(A) and posterior commissure 120(B).

Further, the heart 100 includes the aortic valve 110, which separates the left ventricle 102 from the aorta 122. The aortic valve 110 generally has three cusps/leaflets, wherein each one can have a crescent-type shape. The aortic valve 110 is configured to open during systole to allow blood leaving the left ventricle 102 to enter the aorta 114, and close during diastole to prevent blood from leaking back into the left ventricle 102. The heart 100 also includes the tricuspid valve (not shown), which separates the right atrium from the right ventricle. The tricuspid valve can generally have three cusps or leaflets and can generally close during ventricular contraction (e.g., systole) and open during ventricular expansion (e.g., diastole). Moreover, the heart 100 includes the pulmonary valve (not illustrated), which separates the right ventricle from the pulmonary artery and can be configured to open during systole so that blood can be pumped toward the lungs, and close during diastole to prevent blood from leaking back into the heart 100 from the pulmonary artery. The pulmonary valve generally has three cusps/leaflets, wherein each one can have a crescent-type shape.

The atrioventricular (e.g., mitral and tricuspid) heart valves are generally associated with a sub-valvular apparatus, including a collection of chordae tendineae and papillary muscles securing the leaflets of the respective valves to promote and/or facilitate proper coaptation of the valve leaflets and prevent prolapse thereof. For example, the mitral valve 108 can be associated with chordae tendineae 116 and papillary muscles 118. The papillary muscles 118 can generally comprise finger-like projections from the ventricle walls. Chordae tendineae generally keep the valve leaflets from opening in the wrong direction, thereby preventing blood to flow back to the atrium.

### Example Mitral Valve Conditions

Several diseases/conditions can affect the structure and function of the mitral valve. The mitral valve and, less frequently, the tricuspid valve, are prone to deformation and/or dilation of the valve annulus, tearing of the chordae tendineae, and/or leaflet prolapse, which results in valvular insufficiency wherein the valve does not close properly and allows for regurgitation or back flow from the left ventricle into the left atrium. In some instances, deformations in the structure or shape of the mitral or tricuspid valve can be repairable.

Mitral regurgitation is one of the most common valvular malfunctions in the adult population, and typically involves the elongation or dilation of the posterior two-thirds of the mitral valve annulus, the section corresponding to the posterior leaflet. The most common etiology of systolic mitral regurgitation is myxomatous degeneration, also termed mitral valve prolapse (29% to 70% of cases), which afflicts about 5 to 10 percent of the population in the U.S. Women are affected about twice as often as men. Myxomatous degeneration has been diagnosed as Barlow's syndrome, billowing or ballooning mitral valve, floppy mitral valve, floppy-valve syndrome, prolapsing mitral leaflet syndrome, or systolic click-murmur syndrome. The symptoms can include palpitations, chest pain, syncope or dyspnea, and a mid-systolic click (with or without a late systolic murmur of mitral regurgitation). These latter symptoms are typically seen in patients with Barlow's syndrome. Some forms of mitral valve prolapse seem to be hereditary, though the condition has been associated with Marfan's syndrome, Grave's disease, and other disorders.

Myxomatous degeneration involves weakness in the leaflet structure, leading to thinning of the tissue and loss of coaptation. Barlow's disease is characterized by myxoid degeneration and can appear early in life, often before the age of fifty. In Barlow's disease, one or both leaflets of the mitral valve protrude into the left atrium during the systolic phase of ventricular contraction. The valve leaflets are thick with considerable excess tissue, producing an undulating pattern at the free edges of the leaflets. The chordae are thickened, elongated and may be ruptured. Papillary muscles are occasionally elongated. The annulus is dilated and sometimes calcified. Some of these symptoms are present in other pathologies as well and, therefore, the present application may refer to myxoid degeneration, which is the common pathologic feature of the various diagnoses, including Barlow's syndrome.

Other causes of mitral regurgitation include ischemic heart disease with ischemic mitral regurgitation (IMR), dilated cardiomyopathy (in which the term “functional mitral regurgitation” (FMR) is used), rheumatic valve disease, mitral annular calcification, infective endocarditis, fibroelastic deficiency (FED), congenital anomalies, endocardial fibrosis, and collagen-vascular disorders. IMR is a specific subset of FMR, but both are usually associated with morphologically normal mitral leaflets. Thus, the types of valve disease that lead to regurgitation are varied and present vastly differently.

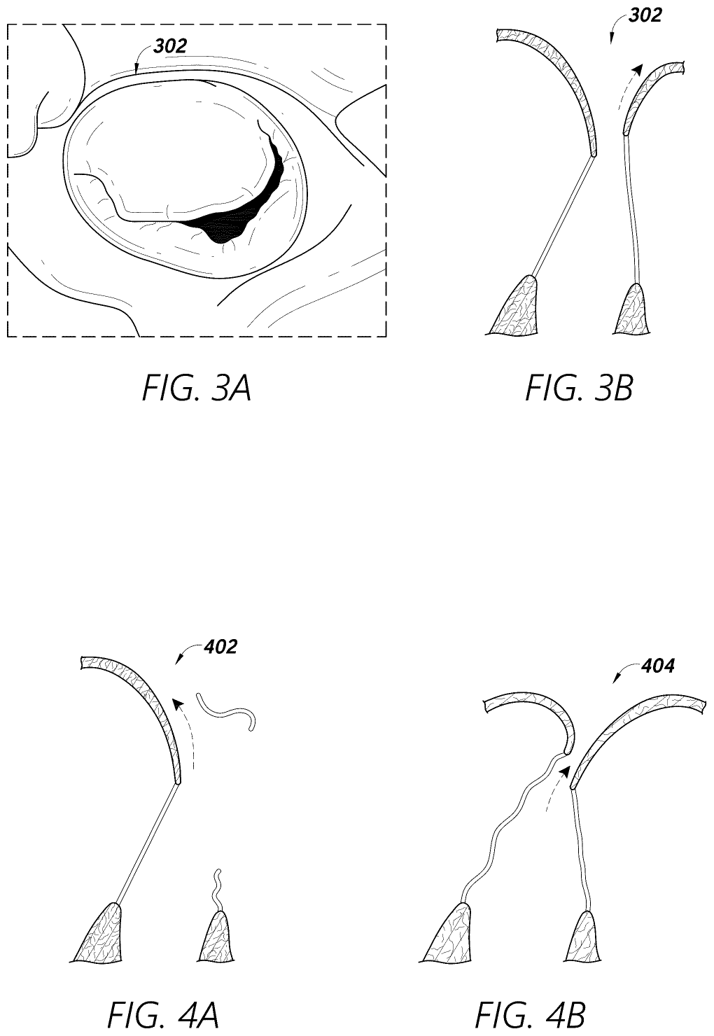

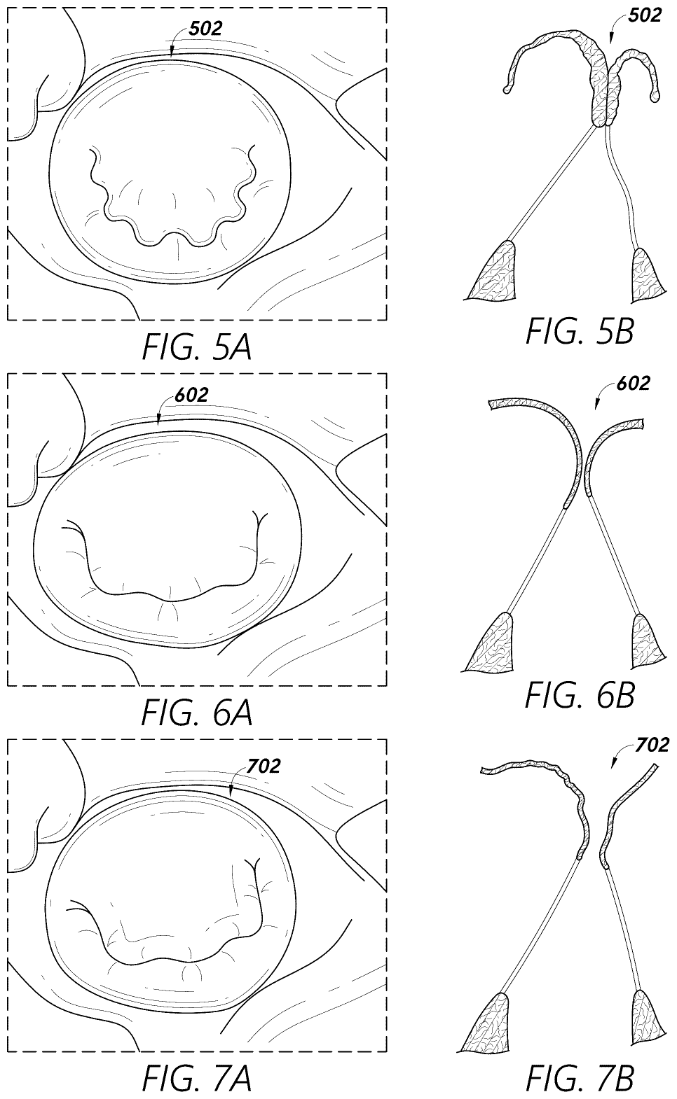

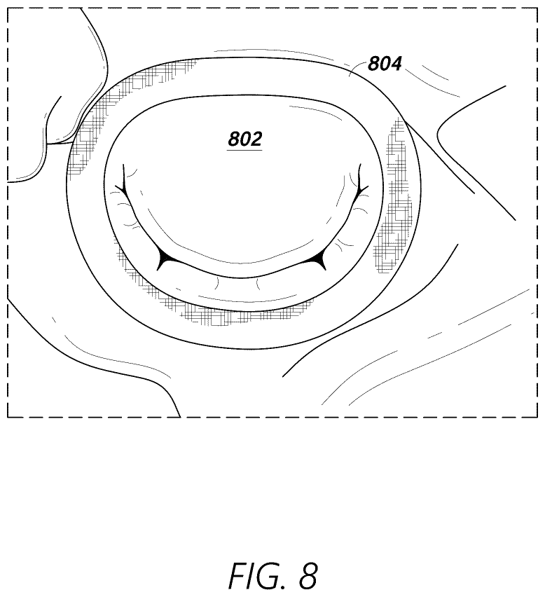

FIGS. 3-7 illustrate various example disease states in cross-sectional and some top/surgeon views of the mitral valve. FIGS. 3A and 3B show a mitral valve 302 where the annulus is dilated and deformed causing mitral regurgitation. FIGS. 4A and 4B illustrate mitral valves 402, 404 with ruptured and elongated chordae, respectively, both causing mitral regurgitation. FIGS. 5A and 5B show a mitral valve 502 with symptoms of Barlow's disease with excess tissue and irregularly thickened leaflets. Barlow's disease is seen most often in the young population and can have a long-lasting evolution before the onset of valve regurgitation. FIGS. 6A and 6B are views of a mitral valve 602 having fibro-elastic deficiency with thinned leaflets and with excess tissue. Fibro-elastic deficiency, first described by Carpentier, is usually seen in more elderly people, and can have a short-lasting evolution before valve regurgitation. The anatomical characteristics include a moderately enlarged kidney shaped valvular orifice without excess leaflet tissue. The leaflet tissue displays a degeneration of the fibro-elastic bundles. Further, FIGS. 7A and 7B illustrate the morphology of a mitral valve 702 in Marfan's disease with excess and thin tissue and elongated chordae. Marfan's is a genetic disorder that can be seen at any age. It has a long-lasting evolution before the onset of regurgitation. The annulus can be severely dilated and deformed, the chordae elongated, and the leaflets thin and degenerative.

As shown from the mitral valves of FIGS. 3-7, many conditions lead to regurgitation and/or other issues. At a structural level, four general types of structural changes of the mitral valve apparatus can cause regurgitation: leaflet retraction from fibrosis and calcification, annular dilation, chordal abnormalities (including rupture, elongation, shortening, or apical tethering or “tenting” as seen in FMR and IMR), and possibly papillary muscle dysfunction.

Example Heart Valve with Annuloplasty Ring

Various techniques/procedures may be used to repair diseased or damaged heart valves, such as mitral and tricuspid valves. These include, but are not limited to, annuloplasty (e.g., contracting/reinforcing the valve annulus to restore the proper size and/or shape of the valve), quadrangular resection of the leaflets (e.g., removing tissue from enlarged or misshapen leaflets), commissurotomy (e.g., cutting the valve commissures to separate the valve leaflets), shortening and transposition of the chordae tendineae, reattachment of severed chordae tendineae or papillary muscle tissue, and decalcification of valve and annulus tissue.

FIG. 8 illustrates an example mitral valve 802 with an annuloplasty ring 804 implanted thereon in an attempt to restore proper function of the mitral valve 802. In examples, the aim of an annuloplasty ring is to restore the shape of the mitral annulus or, in some conditions, to overcorrect the shape by pulling a segment of the annulus inward. In this example, the mitral valve 802 had a deformed annulus leading to regurgitation and the annuloplasty ring 804 is implanted to restore the mitral valve 802 to the normal shape, as shown. The annuloplasty ring 804 can be representative of any of the annuloplasty rings discussed herein. The annuloplasty ring 804 can be sutured to the deformed annulus or attached in another manner. The annuloplasty ring 804 can include a covering (e.g., fabric covering) over a structural interior support or body. In some cases, a suture-permeable interface fills a space between the covering and the interior body. The annuloplasty ring 804 can have a closed or open periphery. Here, the annuloplasty ring 804 is illustrated with a particular form; however, the annuloplasty ring 804 can include other forms, such as other shapes, sizes, materials, and so on, as discussed in further detail below.

Although various techniques/procedures are discussed herein in the context of mitral valves, the techniques/procedures can be applicable to other types of heart valves and/or anatomical structures/features.

### Example Architecture

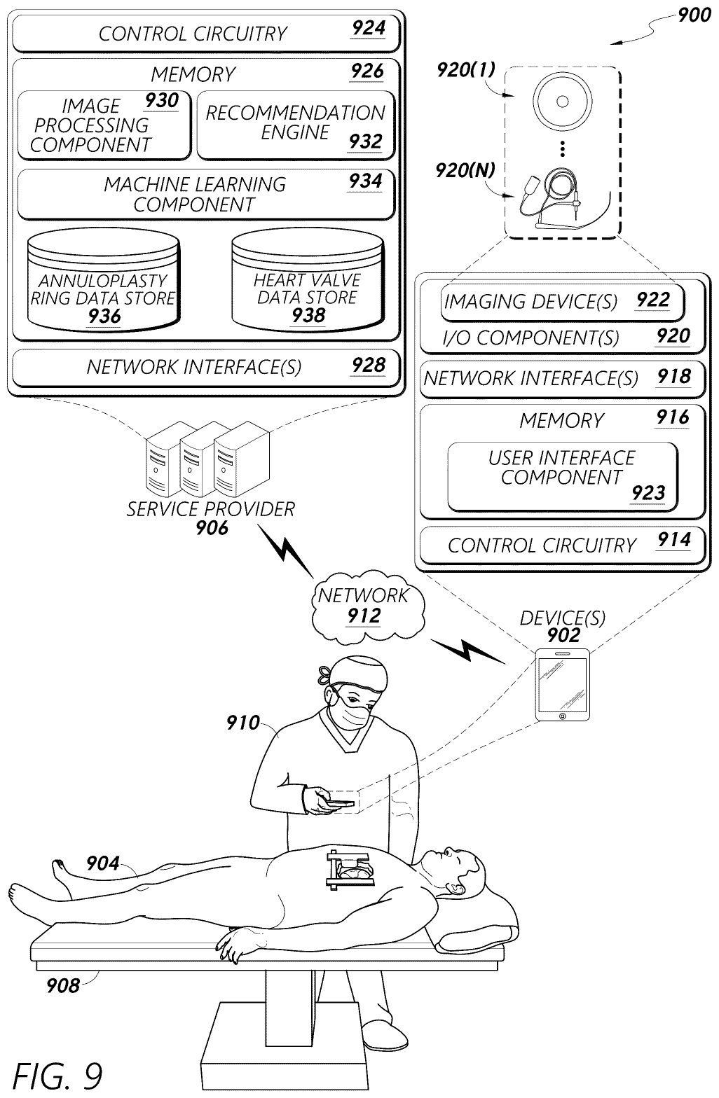

FIG. 9 illustrates an example architecture 900 to implement one or more of the techniques discussed herein. The architecture 900 includes one or more devices 902 (referred to as “the device 902” for ease of discussion) configured to capture/receive data depicting a heart valve of a patient 904 and interface with a user and a service provider 906 configured to process the data to determine/recommend an annuloplasty ring to implant at the heart valve. The following discussion illustrates systems, devices, and methods with reference to a mitral valve, although the teachings are also applicable to any heart valve undergoing annuloplasty, for example, a tricuspid valve. For example, the patient 904 can be positioned on a table 908 for an annuloplasty procedure, wherein a physician 910 implants an annuloplasty ring at a mitral valve or another heart valve of the patient 904. The physician 910 can use the device 902 during the procedure (or before) to capture image data depicting a mitral valve of the patient's heart and to send the image data to the service provider 906. The service provider 906 can perform one or more techniques, such as image processing, to identify one or more measurements and/or other characteristics of the heart valve. Based on the measurement(s) and/or characteristic(s), the service provider 906 can determine an annuloplasty ring that is best suited for the mitral valve of the patient 904, such as a type of annuloplasty ring to implement. The service provider 906 can send data to the device 902 identifying the annuloplasty ring (e.g., a recommendation). The device 902 can output the recommendation to the physician 910, so that the physician 910 can select the appropriate annuloplasty ring for the patient 904. The service provider 906 can also be configured to perform one or more machine learning techniques to generate a model that is configured to identify an annuloplasty ring that is suitable for a set of heart valve characteristics. The device 902 and the service provider 906 can be configured to communicate over one or more networks 912.

Although various functions/operations are discussed as being implemented by a particular device (e.g., the device 902 or the service provider 906), such functions/operations can be divided in other manners. For example, functions discussed as being performed by the service provider 906 can alternatively, or additionally, be implemented by the device 902, and vice versa. Moreover, the functions that are discussed as being performed by the service provider 906 can be implemented across any number of computing devices. For instance, recommendation techniques can be implemented by one or more first computing devices and machine learning techniques can be implemented by one or more second computing devices. Further, for ease of discussion, various acts are discussed as being performed by the physician 190; however, the acts can be performed by another user, such as a user under the direction of the physician 910, a technician, a nurse, and/or any other user.

In examples, an annuloplasty procedure includes a surgical procedure where the patient's chest is cut open to access the heart of the patient (e.g., the heart is visible to the physician). In such procedures, the patient's heart is generally stopped and the patient is connected to a cardiopulmonary bypass machine (also referred to as “a heart-lung machine”) (not shown in FIG. 9) that is configured to take over the function of the patient's heart and lungs. Such procedures are often referred to as “on-pump” or “open heart” procedures. In one example, the patient's heart is stopped, and the physician accesses the mitral valve through the left atrium. Although various annuloplasty procedures are discussed herein in the context of an open-heart surgery, the procedures can be implemented in other manners, such as a minimally invasive procedure where the heart is accessed through a small incision, an off-pump procedure where the heart is still beating and not connected to a heart-lung machine, and so on. In one example of a minimally invasive procedure (or a minimally invasive portion of a surgical procedure), an endoscope or another medical instrument can the inserted through a small incision in the patient and navigated to access the target anatomy for the surgery. Further, in some instances, the techniques discussed herein can be applicable to other types of medical procedures, such as a resection procedure (e.g., removing tissue from enlarged or misshapen leaflets), commissurotomy procedure (e.g., cutting the valve commissures to separate the valve leaflets), chordae tendineae procedure, a procedure to decalcify valve/annulus tissue, or any other procedure relating to a heart valve or other anatomy.

The device 902 can be implemented as one or more computing devices, such as one or more desktop computers, laptops computers, servers, smartphones, electronic reader devices, mobile handsets, personal digital assistants, portable navigation devices, portable gaming devices, tablet computers, wearable devices (e.g., a watch, optical head-mounted display, etc.), portable media players, televisions, set-top boxes, cameras, projectors, medical monitors, and so on. In some examples, the one or more computing devices are implemented as local resources that are located locally relative to the patient 904.

As illustrated, the device 902 can include one or more of the following components, devices, modules, and/or units, either separately/individually and/or in combination/collectively: control circuitry 914, memory/data storage 916, one or more network interfaces 918, and one or more I/O components 920. Although certain components of the device 102 are illustrated in FIG. 9, it should be understood that additional components not shown can be included in examples in accordance with the present disclosure. Furthermore, certain of the illustrated components can be omitted in some examples. Although the control circuitry 914 is illustrated as a separate component in the diagram of FIG. 9, it should be understood that any or all of the remaining components of the device 102 can be embodied at least in part in the control circuitry 914. That is, the control circuitry 914 can include various devices (active and/or passive), semiconductor materials and/or areas, layers, regions, and/or portions thereof, conductors, leads, vias, connections, and/or the like, wherein one or more of the other components of the device 102 and/or portion(s) thereof can be formed and/or embodied at least in part in/by such circuitry components/devices.

The various components of the device 102 can be electrically and/or communicatively coupled using certain connectivity circuitry/devices/features, which may or may not be part of the control circuitry 914. For example, the connectivity feature(s) can include one or more printed circuit boards configured to facilitate mounting and/or interconnectivity of at least some of the various components/circuitry of the device 102. In some examples, two or more of the control circuitry 914, memory/data storage 916, one or more network interfaces 918, and one or more I/O components 920 can be electrically and/or communicatively coupled to each other.

The one or more network interfaces 918 can be configured to communicate with one or more devices/systems over the one or more networks 912. For example, the one or more network interfaces 918 can send/receive data in a wireless and/or wired manner over a network, such as image data, user interface data, and so on. The one or more networks 912 can include one or more local area networks (LAN), wide area networks (WAN) (e.g., the Internet), personal area networks (PAN), body area networks (BAN), etc. In some examples, the one or more network interfaces 918 can implement a wireless technology such as Bluetooth, Wi-Fi, near field communication (NFC), or the like.

The one or more I/O components 920 can include a variety of components to receive input and/or provide output, such as to interface with a user. The one or more I/O components 920 can be configured to receive touch, speech, gesture, or any other type of input. Further, the one or more I/O components 920 can be configured to output display data, audio data, haptic feedback data, or any other type of output data. The one or more I/O components 920 can include one or more displays (sometimes referred to as “one or more display devices”), touchscreens, touch pads, controllers, mice, keyboards, wearable devices (e.g., optical head-mounted display), virtual or augmented reality devices (e.g., head-mounted display), speakers configured to output sounds based on audio signals, microphones configured to receive sounds and generate audio signals, and so on. The one or more displays can include one or more liquid-crystal displays (LCD), light-emitting diode (LED) displays, organic LED displays, plasma displays, electronic paper displays, and/or any other type(s) of technology. In some examples, the one or more displays include one or more touchscreens configured to receive input and/or display data.

As shown, the one or more I/O components 920 can include one or more imaging devices 922 configured to capture/generate image data and/or 2D/3D representations of an environment. The one or more imaging devices 922 can include one or more cameras, range/depth sensors/cameras (e.g., structured-light scanners, time-of-flight cameras, Lidar sensors, etc.), echocardiography device, computed tomography (CT) or computerized axial tomography (CAT) devices, magnetic resonance imaging (MRI) devices, X-ray devices, ultrasound devices, infrared thermography (IRT) devices, positron-emission tomography (PET) devices, and so on. In some cases, the one or more imaging devices 922 can generate data indicating one or more distances to one or more objects/surfaces in an environment (e.g., depth/range data) and/or indicating a coordinate within a coordinate space (e.g., point cloud data). Moreover, in some examples, the one or more imaging devices 922 can be implemented as or coupled to a medical instrument configured to access an anatomical feature of a patient, such as an endoscope configured to navigate within a patient. FIG. 9 illustrates two example imaging devices; namely, a camera 920(1) and an endoscope 920(N) (where N represents an integer greater than one). However, as noted, other types of imaging devices can be implemented. For ease of discussion, many techniques herein refer to processing image data. However, such techniques can be applicable to any type of data generated by an imaging device or other sensor/device of the device 102.

In the example of FIG. 9, the physician 910 uses the device 902 to capture image data of a heart valve of the patient 904 while the patient's heart is exposed during an open-heart procedure. In particular, the physician 910 captures image data using the one or more imaging devices 922. However, the physician 910 can capture image data in other manners and/or without accessing the internal anatomy of the patient 904 (e.g., gaining surgical access to the heart). In one example, the physician 910 captures image data before a procedure using one or more echocardiography devices, computed tomography (CT) or computerized axial tomography (CAT) devices, magnetic resonance imaging (MRI) devices, X-ray devices, ultrasound devices, infrared thermography (IRT) devices, positron-emission tomography (PET) devices, endoscopes, and so on. In another example, the physician 910 uses one or more of such imaging devices during a procedure without/before cutting into the patient 904 (e.g., pre-operative image data).

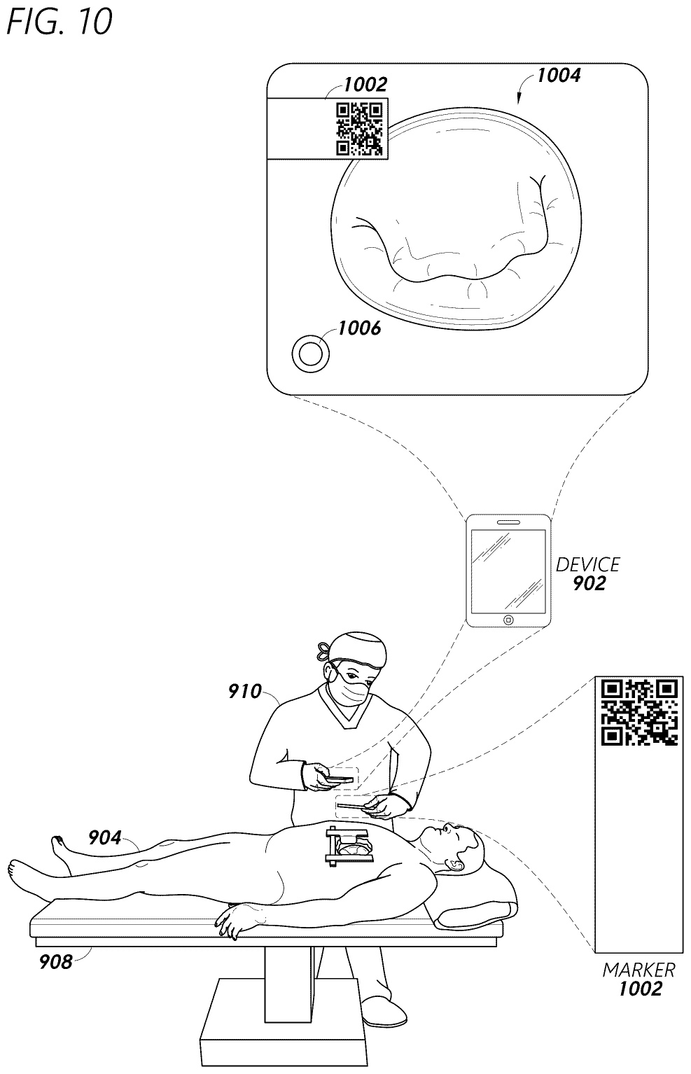

As shown in FIG. 10, in some examples, the physician 910 captures image data of a marker 1002 to assist in identifying one or more measurements/characteristics of the anatomy of the patient 904. The marker 1002 can include a fiduciary marker or another marker/item that can be used as a point of reference for measurement. For example, the marker 1002 can include features arranged in a particular pattern, shape, order, etc. to indicate a particular distance. The marker 1002 can include a quick response (QR) code, a barcode, a ruler, and so on, which may be printed/displayed on an item. In the example of FIG. 10, the physician 910 aims the imaging device 922 of the device 902 at a mitral valve 1004 of the patient 904 and places the marker 1002 within the field-of-view of the imaging device 922 (e.g., within proximity to the mitral valve 1004, adjacent to an annulus of the mitral valve 1004, etc.). The physician 910 then captures image data depicting the mitral valve 1004 and the marker 1002. As shown, the physician 910 can select an icon 1006 to capture the image data.

In returning to FIG. 9, the memory 916 can include a user interface component 923 configured to facilitate various functionality discussed herein. In some examples, the user interface component 923 can include and/or be implemented as one or more executable instructions that, when executed by the control circuitry 914, cause the control circuitry 914 to perform one or more operations. Although many examples are discussed in the context of the user interface component 923 including one or more instructions that are executable by the control circuitry 914, the user interface component 923 can be implemented at least in part as one or more hardware logic components, such as one or more application specific integrated circuits (ASIC), one or more field-programmable gate arrays (FPGAs), one or more program-specific standard products (ASSPs), one or more complex programmable logic devices (CPLDs), and/or the like. Furthermore, although the user interface component 923 is illustrated as being included within the device 102, the user interface component 923 and/or any other component of the device 102 can be implemented at least in part within another device/system, such as the service provider 906.

The user interface component 923 can be configured to interface with the physician 910 and/or another user to provide/receive various input/output. For example, the physician 910 can provide input to capture image data, request that the image data be processed to generate a recommendation regarding an annuloplasty ring, view information regarding a recommended annuloplasty ring, provide input indicating a fit/effectiveness of the annuloplasty ring, and so on. The user interface component 923 can be configured to operate in cooperation with the one or more I/O components 920 and/or other components of the device 102. In some cases, the physician 910 can view image data depicting anatomy of the patient 904 and provide input regarding a characteristic of an anatomical feature, as discussed in further detail below.

The service provider 906 may be implemented as one or more computing devices, such as one or more servers, one or more desktop computers, one or more laptops computers, or any other type of device configured to process data. In some examples, the one or more computing devices are configured in a cluster, data center, cloud computing environment, or a combination thereof. Further, in some examples, the one or more computing devices are implemented as a remote computing resource that is located remotely to the device 102. In other examples, the one or more computing devices of the service provider 906 are implemented as local resources that are located locally at the device 102. Further, in some instances the functions of the service provider 906 and the device 102 can be performed/implemented by a single device.

As illustrated, the service provider 906 can include one or more of the following components, devices, modules, and/or units (referred to herein as “components”), either separately/individually and/or in combination/collectively: control circuitry 924, memory/data storage 926, and one or more network interfaces 928. Although certain components of the service provider 906 are illustrated in FIG. 9, it should be understood that additional components not shown can be included in examples in accordance with the present disclosure. Furthermore, certain of the illustrated components can be omitted in some examples. Although the control circuitry 924 is illustrated as a separate component in the diagram of FIG. 9, any or all of the remaining components of the service provider 906 can be embodied at least in part in the control circuitry 924. That is, the control circuitry 924 can include various devices (active and/or passive), semiconductor materials and/or areas, layers, regions, and/or portions thereof, conductors, leads, vias, connections, and/or the like, wherein one or more of the other components of the service provider 906 and/or portion(s) thereof can be formed and/or embodied at least in part in/by such circuitry components/devices.

The various components of the service provider 906 can be electrically and/or communicatively coupled using certain connectivity circuitry/devices/features, which may or may not be part of the control circuitry 924. For example, the connectivity feature(s) can include one or more printed circuit boards configured to facilitate mounting and/or interconnectivity of at least some of the various components/circuitry of the service provider. In some examples, two or more of the control circuitry 924, memory/data storage 926, and one or more network interfaces 928 can be electrically and/or communicatively coupled to each other.

The one or more network interfaces 928 can be configured to communicate with one or more devices/systems over the one or more networks 912. For example, the one or more network interfaces 928 can send/receive data in a wireless and/or wired manner over a network, such as image data, user interface data, recommendation data, and so on.

As shown, the memory 926 can include an image processing component 930, a recommendation engine 932, and a machine learning component 934 configured to facilitate various functionality discussed herein. In examples, one or more of the elements 930-934 can include and/or be implemented as one or more executable instructions that, when executed by the control circuitry 924, cause the control circuitry 924 to perform one or more operations. Although many examples are discussed in the context of one or more instructions that are executable by the control circuitry 924, the image processing component 930, the recommendation engine 932, and/or the machine learning component 934 can be implemented at least in part as one or more hardware logic components, such as one or more application specific integrated circuits (ASIC), one or more field-programmable gate arrays (FPGAs), one or more program-specific standard products (ASSPs), one or more complex programmable logic devices (CPLDs), and/or the like. Furthermore, although the image processing component 930, the recommendation engine 932, and the machine learning component 934 are illustrated as being included within the service provider 906, any of such elements and/or any other component of the service provider 906 can be implemented at least in part within another device/system, such as the device 102. The memory 926 can also include an annuloplasty ring data store 936 to store annuloplasty ring data and a heart valve data store 938 to store heart valve data, as discussed in further detail below.

The image processing component 930 can be configured to analyze image data and/or other data depicting one or more anatomical features. For example, the image processing component 930 can receive image data from the device 902 and process the image data using one or more image processing techniques to automatically identify image-based features within the one or more images and/or classify the one or more image-based features as anatomical features. In some instances, one or more image processing techniques can include detection that seeks to identify one or more image features within an image (e.g., edges, corners, blobs, ridges, and so on), tracking that seeks to track one or more image features across images/frames, and/or classification that seeks to classify the one or more image features into one or more categories. The one or more image processing techniques can include various forms of image processing, such as curvature detection, feature extraction, filtering, contrast detection (e.g., detecting features based on differences in contrast in the image), or any other techniques. In some examples, image data represents multiple images (e.g., video data, multiple still images at different times, etc.), while in other instances the image data represents a single image. The image processing component 930 can also be configured to determine a measurement/characteristic of an anatomical feature of a patient based on the one or more image-based features. The image processing component 930 can store heart valve data in the heart valve data store 938 indicating measurements/characteristics of the anatomical feature. In examples, the image processing component 930 can use one or more models/algorithms, such as a machine-trained model, user-trained model, or another model that has been trained to analyze image data, classify features in the image data, and/or determine a measurement/characteristic of an anatomical feature. In some instances, a machine-trained model is trained using artificial intelligence (e.g., machine learning).

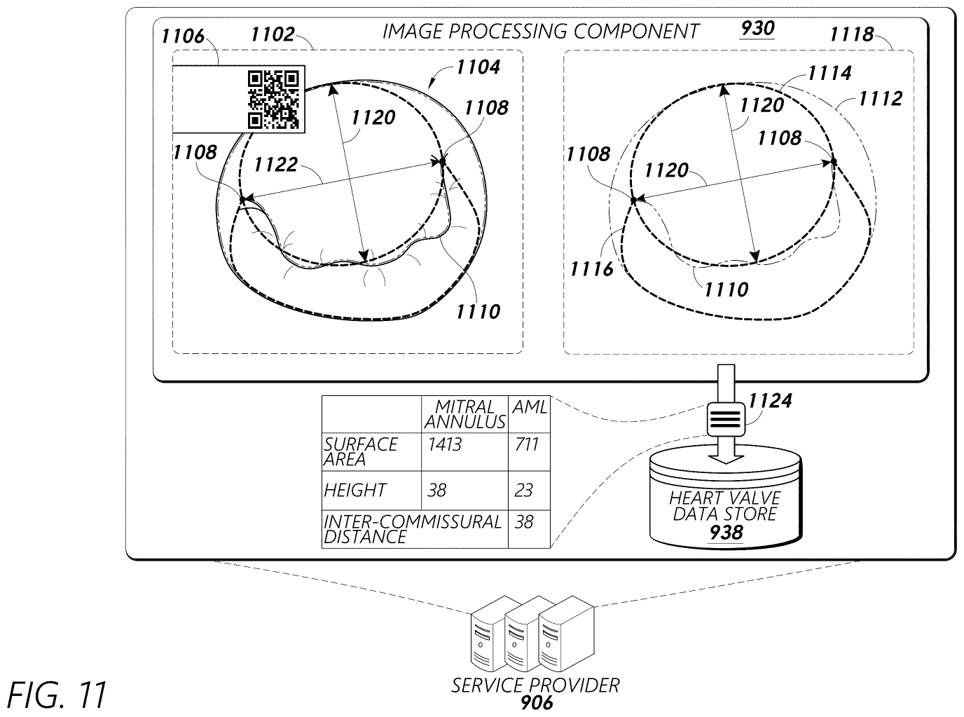

In one illustration, as shown in FIG. 11, the image processing component 930 analyzes image data 1102 depicting a mitral valve 1104 and a marker 1106. Here, the mitral valve 1104 depicts an unhealthy mitral valve. Although the marker 1106 is shown in this example, the marker 1106 may not be used. The image processing component 930 can identify one or more features in the image data 1102 representing one or more anatomical features of the mitral valve 1104. For instance, the image processing component 930 can identify image features representing a leaflet, coaptation of leaflets, a portion of a leaflet (e.g., scalloped/non-scalloped portions), a commissure (e.g., an area where leaflets abut), a valve annulus, an opening between leaflets (e.g., a gap due to improper coaptation), and so on. In the example shown in FIG. 11, the image processing component 930 identifies commissure features 1108 representing commissures of the mitral valve 1104, a coaptation feature 1110 representing a contour/line/area of coaptation of leaflets of the mitral valve 1104, and an annulus feature 1112 representing the mitral valve annulus. In some cases, the image processing component 930 can generate contours/lines representing/connecting various identified features. For example, based on the commissure features 1108, the coaptation features 1110, and/or the annulus feature 1112, the image processing component 930 can generate a contour/line 1114 that generally represents an outer edge/border of the anterior leaflet of the mitral valve 1104 and a contour/line 1116 that generally represents an outer edge/border of the posterior leaflet of the mitral valve 1104. The depiction at 1118 illustrates various identified/generated image features of the image data 1102 removed from/without the image data 1102.

In continuing with the illustration of FIG. 11, the image processing component 930 can use any of the information noted above to determine various characteristics/measurements of the mitral valve 1104. Such characteristics/measurements can include: a surface area of the mitral annulus (e.g., area within the annulus feature 1112) (also referred to as “the surface area of the mitral valve”), a surface area of the anterior/posterior mitral leaflet (e.g., based on the contour line 1114/1116 and/or the coaptation feature 1110), a height of the mitral annulus, a height of the anterior/posterior leaflet (e.g., a height 1120 of the anterior mitral leaflet, which can be based on the contour line 1114), an inter-commissural distance 1122 (e.g., a distance between commissure features 1108), a distance/circumference of the mitral annulus (e.g., based on the annulus feature 1112), a length/width of the anterior/posterior mitral leaflet (e.g., based on the contour line 1114/1116), an amount of coaptation gap due to improper coaptation of leaflets (e.g., based on the coaptation feature 1110), a diameter of the mitral annulus (e.g., based on the annulus feature 1112), a number of clefts, a comparison/difference of the surface area of the anterior/posterior leaflet to the surface area of the mitral annulus, a comparison/difference of the surface area of the anterior leaflet to the posterior leaflet, and so on. In some cases, the image processing component 930 uses information indicated by the marker 1106 (e.g., a predetermined distance) to determine a characteristic/measurement of the mitral valve 1104. However, the marker 1106 may not be used in some cases. In any event, the image processing component 930 can store heart valve data 1124 indicating the characteristics/measurements of the mitral valve 1104 in the heart valve data store 938, as shown. The heart valve data store 938 can store heart valve data for any number of patients, such as overtime as patients undergo surgery and/or heart imaging.

In another illustration (not shown in FIG. 9), a user can view image data depicting anatomy of a patient and provide input regarding a characteristic/measurement of an anatomical feature. In one example, the physician 910 (or another user, such as someone at the site of the surgery or offsite) can interface with the device 102 to designate an image feature as representing a particular anatomical feature (e.g., draw a line/point/circle on a touch screen to identify/trace a leaflet, edge of a leaflet, commissure, annulus, etc.). In another example, a user can designate a first point/location on an image and a second point/location in the image and provide input requesting that a distance be calculated between the first point/location and the second point/location. The user can also provide input to label the distance. A user can provide input to determine/label any of the characteristics/measurements discussed herein. The image processing component 930 can use input provided by a user to evaluate/analyze one or more images and/or store data regarding one or more characteristics/measurements of one or more anatomical features in the heart valve data store 938.

In some instances, the image processing component 930 (and/or a component of the device 102, such as the imaging device 922) is configured to generate an n-dimensional representation of an anatomical feature. The n-dimensional representation can include 2D/3D representation, such as a surface model, solid model, wire-frame, point cloud, and so on. Here, the image processing component 930 can be configured to analyze the n-dimensional representation to determine a characteristic/measurement of an anatomical feature and/or store heart valve data or other anatomical feature data in the heart valve data store 938. Further, in some cases, data regarding an n-dimensional representation can be stored in the heart valve data store 938.

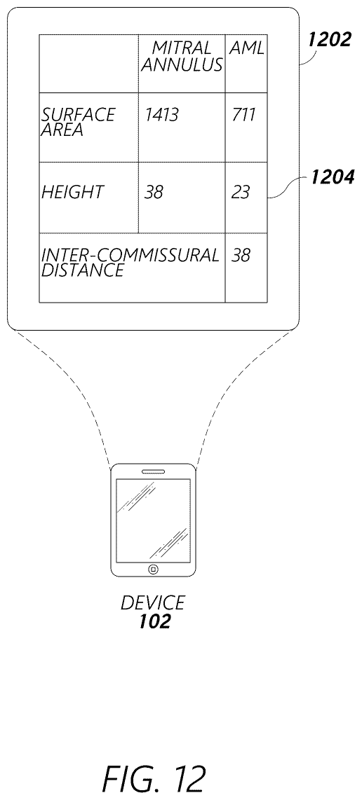

In examples, data indicating a characteristic/measurement of an anatomical feature can be provided to a user. For instance, as shown in FIG. 12 the device 102 can provide an interface 1202 with information 1204 indicating one or more measurements of a mitral valve of a patient. This can allow the physician 910 and/or another user to better understand the mitral valve of the patient. Although the information 1204 is related to the mitral valve, information can be provided regarding any anatomical feature and/or any measurement/characteristic.

The recommendation engine 932 can be configured to determine/select an annuloplasty ring that is most appropriate for a situation. For example, in returning to FIG. 9, the recommendation engine 932 can use heart valve data for the patient 904 to determine a type of annuloplasty ring that is best suited for implantation at a mitral valve of the patient 904. The recommendation engine 932 can also analyze annuloplasty ring data stored in the annuloplasty ring data store 936 to determine the type of annuloplasty ring. In examples, the recommendation engine 932 can analyze multiple characteristics/measurements of an anatomical feature and/or perform such analysis for multiple annuloplasty rings to thereby identify a most appropriate annuloplasty ring from among many annuloplasty rings that are available for implantation. Upon identifying an annuloplasty ring for a situation, the recommendation engine 932 can generate and/or send output data to the device 102 and/or another device identifying the annuloplasty ring (e.g., recommend that the annuloplasty ring be used). This can enable the physician 910 to select an annuloplasty ring that is most appropriate for the particular anatomy of the patient 904.

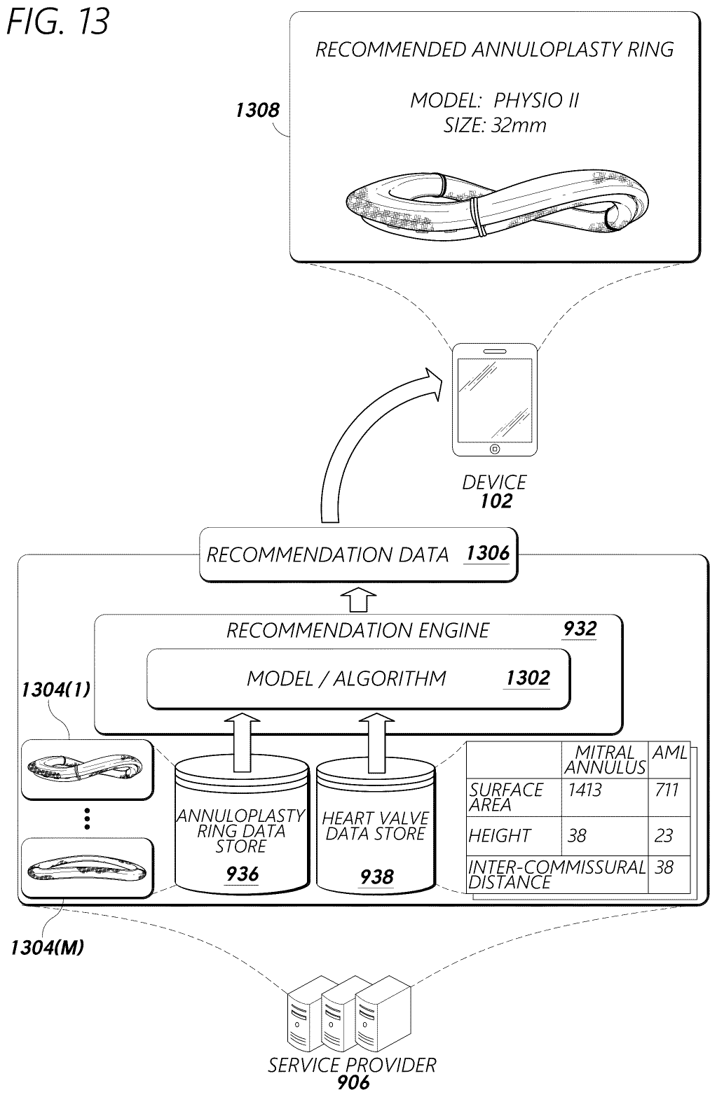

In one illustration, as shown in FIG. 13, the recommendation engine 932 can use a model/algorithm 1302 to determine an annuloplasty ring that is most appropriate for implantation at a heart valve. The recommendation engine 932 can receive annuloplasty ring data from the annuloplasty ring data store 936. The annuloplasty ring data can indicate one or more characteristics of one or more annuloplasty rings, such as a size of an annuloplasty ring(s), a shape of the annuloplasty ring(s), a type of suture feature of the annuloplasty ring(s), a location on the annuloplasty ring to suture the annuloplasty ring to the heart valve(s), whether the annuloplasty ring is a closed or open ring(s), a flexibility of the annuloplasty ring(s), a material of the annuloplasty ring, a structure of an inner ring body, a number of inner ring bodies, a structure of an outer covering, any dimension(s) of the annuloplasty ring, and so on. A characteristic of an annuloplasty ring can indicate the type of annuloplasty ring. The annuloplasty ring data store 936 can store data for multiple annuloplasty rings 1304(1)-1304(M) (where M represents an integer greater than one), such as multiple types of annuloplasty rings that may be available for implantation, multiple annuloplasty rings that are available to a physician during a procedure (e.g., in stock at a hospital), and so on. Further, the recommendation engine 932 can receive heart valve data from the heart valve data store 938. The heart valve data can indicate one or more characteristics/measurements of a heart valve of a patient. The model/algorithm 1302 receive the heart valve data and the annuloplasty ring data as input data.

In continuing with this illustration, the recommendation engine 932 can use the input data to determine, from among the multiple annuloplasty rings 1304, an annuloplasty ring for the characteristics/measurements of the heart valve. The model/algorithm 1302 can output data indicating the annuloplasty ring to use and/or one or more characteristics of the annuloplasty ring. In some examples, the output data indicates one or more confidence values/scores for one or more annuloplasty rings. The recommendation engine 932 can then send recommendation data 1306 (e.g., user interface data) to the device 102 for output to a user. The recommendation data 1306 can indicate one or more characteristics of an annuloplasty ring(s) and/or confidence values/scores of the annuloplasty ring(s). As shown in FIG. 13, the recommendation data 1306 can be used to output information regarding the annuloplasty ring(s) via a user interface 1308 of the device 102. Here, the information indicates a model/name of the annuloplasty ring (e.g., “Physio II”) and a size of the annuloplasty ring (e.g., 32 mm). A physician can view the information (e.g., the recommendation) and, if desired, select the annuloplasty ring for implantation. Although a model/name and size of a recommended annuloplasty ring are presented within the user interface 1308 in this example, the user interface 1308 can provide any information regarding an annuloplasty ring. For example, the recommendation engine 932 can provide information indicating a size of an annuloplasty ring without identifying a model/identifier, a model of an annuloplasty ring without identifying a size, a shape of an annuloplasty ring, an open or closed characteristic of an annuloplasty ring, and so on.

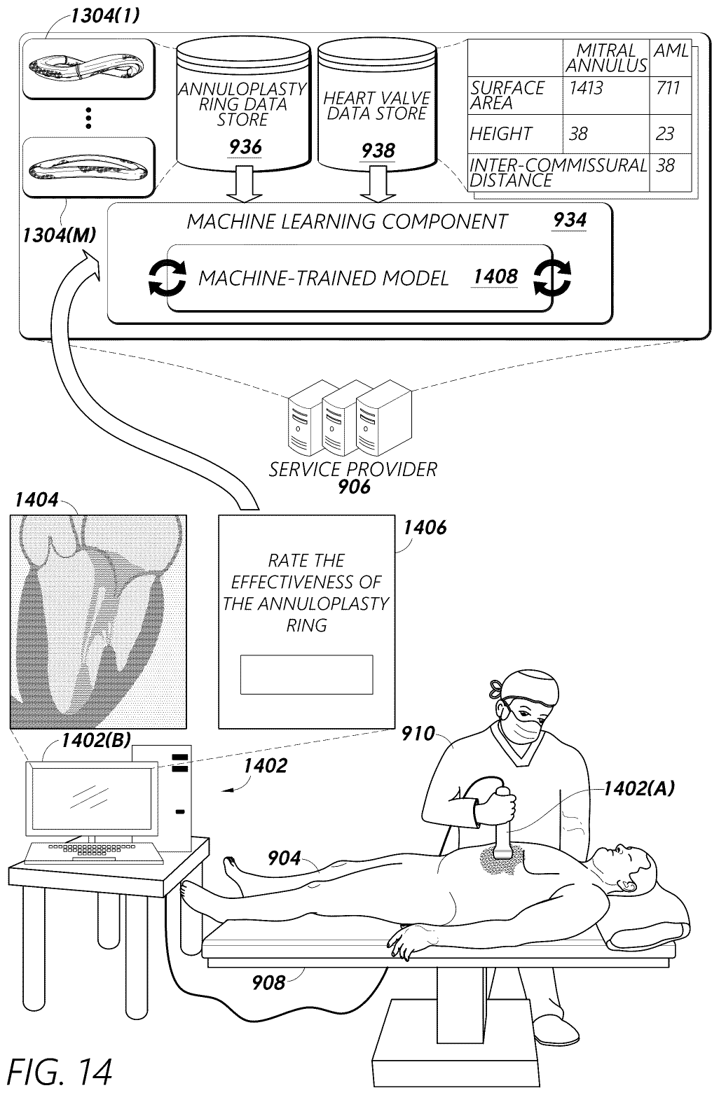

In some examples, the recommendation engine 932 can determine an annuloplasty ring by ranking multiple annuloplasty rings based on an estimated fit. For instance, the model/algorithm 1302 can compare a characteristic/measurement of a heart valve to a characteristic of an annuloplasty ring to determine a fit value. The model/algorithm 1302 can perform such comparison for multiple characteristics/measurements of a heart valve and/or weight each resulting fit value. The fit values (in weighted or non-weighted form) can be aggregated to determine an overall score for the annuloplasty ring. Such processing can be performed for multiple annuloplasty rings to determine multiple scores for the annuloplasty rings, respectively. The model/algorithm 1302 can then rank the annuloplasty rings based on the scores and determine/select an annuloplasty ring that ranks the highest (or lowest, in some cases). Further, in some examples, the recommendation engine 932 can determine an annuloplasty ring based on a machine-/user-trained model. For instance, as discussed in further detail below in reference to FIG. 14, a model can be trained to determine/select an annuloplasty ring for a set of anatomical characteristics. Such training can use artificial intelligence techniques (e.g., machine learning). In any event, the model can be configured to receive heart valve data and/or annuloplasty ring data as input and provide a recommended annuloplasty ring as output.

In some instances, the recommendation engine 932 generates recommendation data during a procedure based on image data captured during the procedure. Here, the recommendation engine 932 can provide recommendation data in a relatively short period of time (e.g., less than a threshold amount of time) to minimize the amount of time of the procedure, which can avoid complications due to a patient being connected to a heart-lung machine and/or otherwise exposed in a surgical environment. In other instances, the recommendation engine 932 generates recommendation data based on pre-operative data, such as imaging data captured before a procedure (e.g., using 2D or 3D echo data from an echocardiography device or other machine). Here, a physician can prepare in advance for a medical procedure.

In some examples, the recommendation engine 932 can run a simulation to evaluate a fit of an annuloplasty ring for a heart valve. For example, the recommendation engine 932 can select one or more annuloplasty rings (e.g., a predetermined number of annuloplasty rings that are best suited for implantation or any number of available annuloplasty rings) and implement a simulation on each of the one or more annuloplasty rings to determine how the heart valve would function with the respective annuloplasty ring implanted thereon. The simulation can provide an approximated/estimated imitation of an amount of regurgitation, excessive tissue folds into the valve, leaflet coaptation, systolic anterior motion (SAM), heart remodeling, and so on. Based on the simulation, the recommendation engine 932 can select an annuloplasty ring for recommendation, such as an annuloplasty ring that satisfies one or more criteria.

In returning to FIG. 9, the machine learning component 934 can be configured to perform one or more machine learning techniques to learn a type of annuloplasty ring and/or characteristic of an annuloplasty ring that is most appropriate for a situation. For example, the machine learning component 934 can analyze post-operative image data (also referred to as post-procedure image data) depicting an anatomical feature after a medical procedure, data indicating an effectiveness of an implanted annuloplasty ring or another medical implant, and/or pre-operative anatomical feature data indicating/depicting an anatomical feature before a medical procedure (e.g., heart valve data before a procedure, image data before a procedure, etc.). Such data can be referred to as “training data.” In examples, the machine learning component 934 can receive data from a user indicating an effectiveness of an implanted annuloplasty ring and/or automatically determine an effectiveness of an annuloplasty ring, as discussed in further detail below. The machine learning component 934 can train a model (e.g., an artificial neural network or another artificial intelligence model) to generate a machine-trained model, which can be stored in the memory 926. The machine learning component 934 can learn over various patients and/or data sets, such as by determining correlations between particular characteristics/measurements of an anatomical feature and characteristics of annuloplasty rings. In some instances, a different machine-trained model is generated for different contexts, such as different types of medical procedure, anatomical features, and so on.

In one illustration, as shown in FIG. 14, the machine learning component 934 is configured to train a model based on post-operative data from the patient 904 that has undergone an annuloplasty procedure. Here, an annuloplasty ring has been implanted at the mitral valve of the patient 904, and the physician 910 or another user uses an imaging system 1402 to evaluate an effectiveness of the procedure. The imaging system 1402 can be implemented as one or more echocardiography devices, computed tomography (CT) or computerized axial tomography (CAT) devices, magnetic resonance imaging (MRI) devices, X-ray devices, ultrasound devices, infrared thermography (IRT) devices, positron-emission tomography (PET) devices, endoscopes, and so on. As shown, the physician 910 or another user can use a scanning device 1402(A) of the imaging system 1402 to capture/generate image data 1404 (e.g., echocardiogram, CT image, X-ray image, etc.) of the mitral valve. The scanning device 1402(A) can be communicatively coupled to a computing device 1402(B) of the imaging system 1402, which can receive the image data 1404 and/or display the image data 1404 via a display device.

In continuing with this illustration, the physician 910 or another user can view the image data 1404 and evaluate an effectiveness of the annuloplasty ring. For example, the physician 910 can determine if there is any regurgitation (e.g., due to implantation of too large of an annuloplasty ring), if excessive tissue folds into the valve (e.g., due to implantation of too small of an annuloplasty ring), if there is any systolic anterior motion (SAM) of the mitral valve (e.g., obstruction of the anterior leaflet into the outflow track of blood through the aortic valve), if the heart remodels in an undesired manner, if the leaflets properly coapt, and so on. In some instances, the physician 910 can rate the effectiveness of the annuloplasty ring by providing input via a user interface 1406 displayed/output via the computing device 1402(B) or another device. In this example, the physician 910 can provide a score, such as on a scale of 1 to 10, indicating an effectiveness of the procedure. However, the physician 910 can provide other input, such as text/speech input generally describing the state of the mitral valve, input identifying undesired features of the mitral valve (e.g., circling a prolapse/regurgitation location on the image data 1404), and so on. The computing device 1402(B) can send (over a network) a communication/user input data to the service provider 906 indicating the input received from the physician 910.

The machine learning component 934 can update a machine-trained model 1408 based on the user input data. For instance, the machine learning component 934 can learn that the model 1408 selected an improper annuloplasty ring if the physician indicates that the effectiveness of the annuloplasty ring is relatively low. The machine learning component 934 can then update one or more parameters associated with the machine-trained model 1408. In contrast, one or more parameters of the machine-trained model 1408 may not be updated (or updated relatively little) if the physician 910 indicates that the procedure was relatively effective. Such process can be repeated for any number of patients over time so that the machine learning component 934 can learn characteristics of an annuloplasty ring that are most appropriate for a situation.

In another illustration, the machine learning component 934 can train the machine-trained model 1408 based on an automatic analysis of image data captured after a procedure. Here, the machine learning component 934 can analyze the image data to determine regurgitation, excessive tissue folding into the valve, systolic anterior motion (SAM) of the mitral valve, heart remodeling (e.g., by comparison to pre-procedure image data), coaptation of leaflets, and so on. Based on such automatic analysis, the machine learning component 934 can determine an effectiveness of the procedure and update the machine-trained model 1408, if needed, in a similar manner as discussed above.

As noted above, the techniques discussed herein can more accurately determine an annuloplasty ring for a particular context, in comparison to other solutions. For example, the techniques can perform image processing or other techniques to identify one or more characteristics/measurements of a heart valve and use such information to identify an annuloplasty ring. In some cases, multiple characteristics/measurements of a heart valve can be identified and used, which can result in a more accurate selection of an annuloplasty ring, in comparison to other solutions which rely on limited information. Further, the techniques can rely on machine learning to generate a model that is configured to identify an annuloplasty ring for a set of characteristics/measurements. Such model can be trained over various patients and/or data sets to determine correlations between characteristics/measurements and annuloplasty rings. Moreover, the techniques can enable physicians to more consistently select an annuloplasty ring (e.g., avoid disagreement amongst physicians about which annuloplasty ring to use for a particular context).

Although various techniques are discussed herein in the context of an annuloplasty procedure, the techniques can also be applicable to other types of procedures, such as a resection procedure, commissurotomy procedure, chordae tendineae procedure, a procedure to decalcify valve/annulus tissue, or any other procedure relating to a heart valve or other anatomy. In one example, a machine-trained model can be used to recommend an amount of a leaflet to remove and/or where to remove tissue from a leaflet during a resection procedure. In another example, a machine-trained model can be used to recommend a length of the chordae tendineae of a heart valve during a chordae tendineae procedure. In yet another example, a machine-trained model can be used to recommend a type of procedure to perform to repair a heart valve.

The term “control circuitry” can refer to any collection of one or more processors, processing circuitry, processing modules/units, chips, dies (e.g., semiconductor dies including come or more active and/or passive devices and/or connectivity circuitry), microprocessors, micro-controllers, digital signal processors, microcomputers, central processing units, graphics processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry can further comprise one or more, storage devices, which can be embodied in a single memory device, a plurality of memory devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. It should be noted that in examples in which control circuitry comprises a hardware state machine (and/or implements a software state machine), analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

The term “memory” can refer to any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

One or more computer-readable media that can be implemented in accordance with examples of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

### Example Annuloplasty Rings

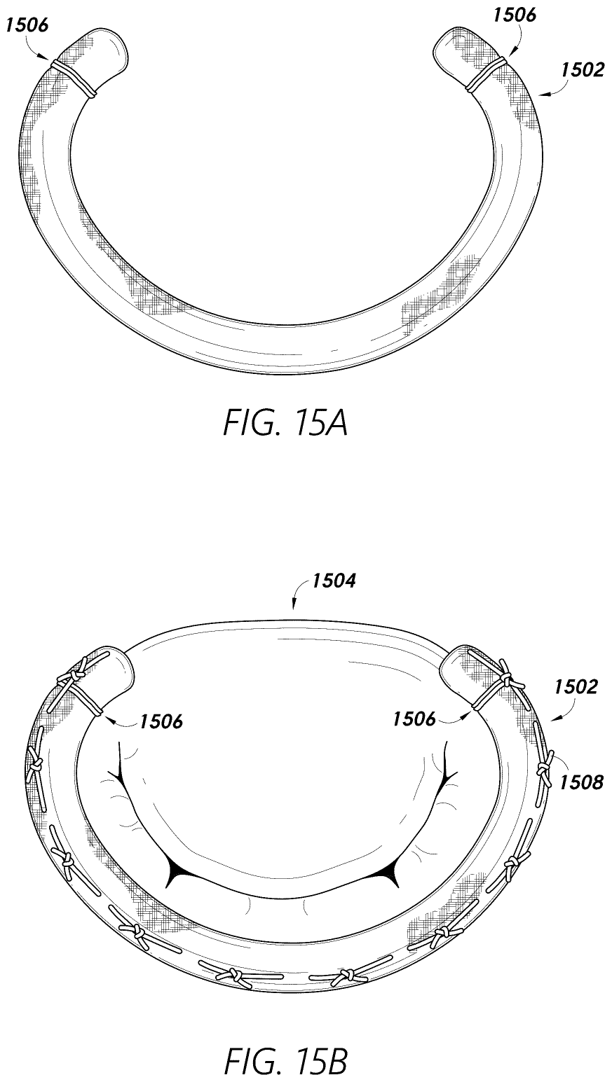

FIGS. 15-19 illustrate example annuloplasty rings that can be implemented in accordance with one or more examples of the present disclosure. These annuloplasty rings are discussed for illustrative purposes, and other types of annuloplasty rings can be implemented. Although various examples are illustrated/discussed in the context of a mitral valve, the annuloplasty rings can be implemented on other types of heart valves. An annuloplasty ring can have any of the characteristics/features discussed below and/or additional characteristics/features (or less than those discussed).

An annuloplasty ring can generally include a ring body/structural interior support and an outer covering disposed over the ring body. In examples, at least a portion of the outer covering includes a suture-permeable material (e.g., fabric) and/or other features to facilitate attachment of the annuloplasty ring to the anatomy, such as using one or more sutures. In some examples, an annuloplasty ring can include a dedicated suture features/cuff, which can be indicated on the annuloplasty ring. The ring body can be formed of one or more bands/structural features. In examples, the ring body can be configured to at least partially resist deformation when subjected to stress imparted thereon by the mitral valve annulus. As such, an annuloplasty ring can be rigid and/or at least partially flexible across at least a portion of the ring, such as an anterior or posterior portion. An annuloplasty ring can have a closed/continuous periphery or an open periphery. In some instances, an annuloplasty ring is symmetrical about an axis, while in other instances an annuloplasty ring is asymmetrical. An annuloplasty ring can have a variety of sizes, such as 24 mm, 26 mm, 28 mm, 30 mm, 32 mm, 34 mm, 36 mm, 38 mm, 40 mm, and so on, which can generally refer to a distance between features that attach near the commissures of the heart valve (e.g., a largest inner distance/diameter, which is near the commissure/trigon attachment points). Further, annuloplasty rings can have a variety of different shapes, which can be based on one or more dimensions of the annuloplasty ring (e.g., height, width, length, thickness, etc.), curvature properties of the annuloplasty ring (e.g., a 2D or 3D bow), and so on. In one illustration, a closed ring has a D-or kidney-shape and/or exhibits a minor/major axis ratio of about 3:4. Some rings are flat or planar, while others exhibit three-dimensional bows.

FIGS. 15A and 15B illustrate an example open/partial annuloplasty ring 1502. FIG. 15A illustrates the annuloplasty ring 1502 by itself, while FIG. 15B illustrates the annuloplasty ring 1502 implanted on a mitral valve 1504. The annuloplasty ring 1502 can be configured to preserve natural flexibility of the anatomy. As shown, the annuloplasty ring 1502 can have an open design in the anterior portion (when implemented in the context of a mitral valve) and/or in the septal portion (when implemented in the context of a tricuspid valve). The annuloplasty ring 1502 can be generally flexible and/or conform to a 3D annular shape of the heart valve. The annuloplasty ring 1502 can provide support against dilation of a heart valve. The annuloplasty ring 1502 can include markers 1506 that can be used to align the annuloplasty ring 1502 with the mitral valve 1504, such as by aligning the markers 1506 with the commissures of the mitral valve 1504. FIG. 15B illustrates sutures/stitches 1508 attaching the annuloplasty ring 1502 to the mitral valve 1504. In this example the sutures 1508 are disposed around a periphery of the annuloplasty ring 1502; however, the sutures 1508 can be positioned at other locations.

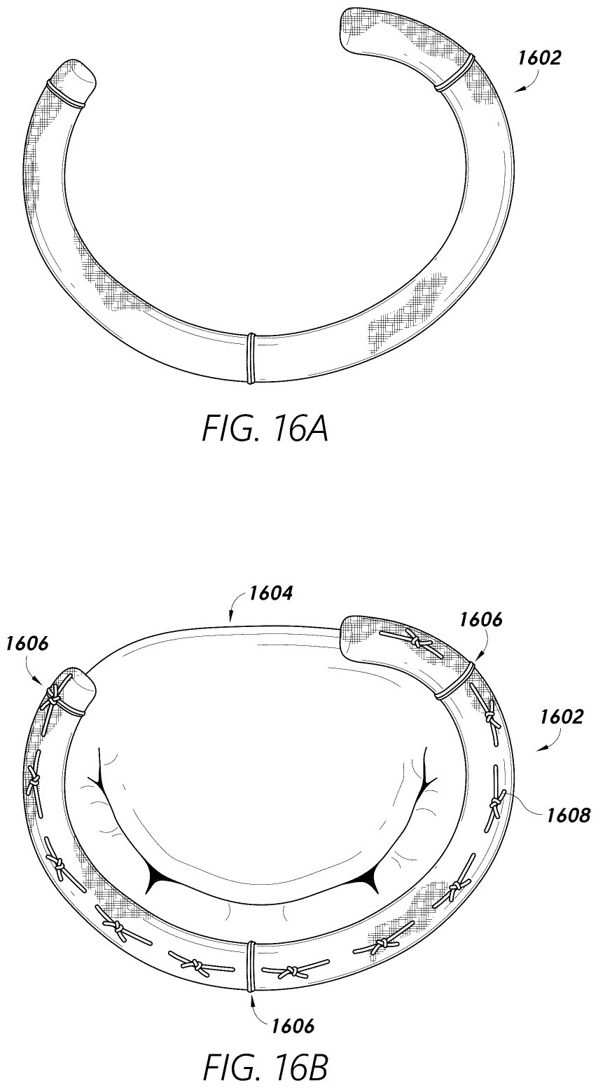

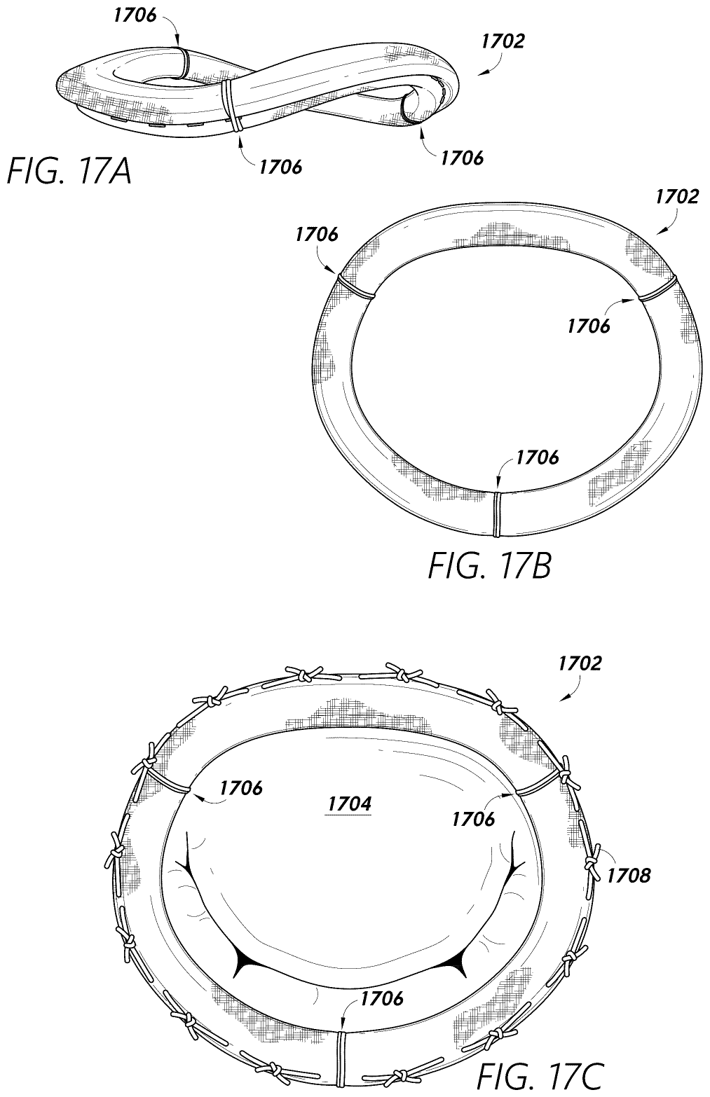

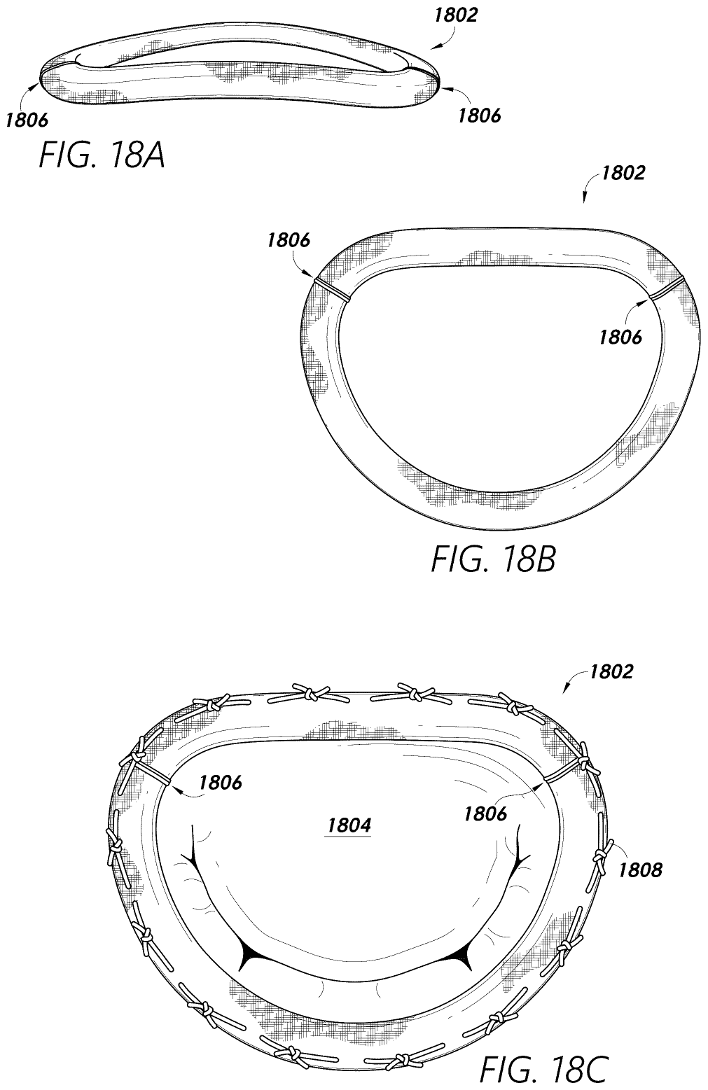

FIGS. 16A and 16B illustrate another example open/partial annuloplasty ring 1602. FIG. 16A illustrates the annuloplasty ring 1602 by itself, while FIG. 16B illustrates the annuloplasty ring 1602 implanted on a mitral valve 1604. The annuloplasty ring 1602 can be configured to perform at least some remodeling of the anatomy while preserving at least some flexibility of the anatomy. The annuloplasty ring 1602 can be implemented as a semi-rigid ring. As shown, the annuloplasty ring 1602 can have an open anterior segment associated with the aorta aorto-mitral curtain. The annuloplasty ring 1602 can be configured such that anterior and posterior saddle shapes correspond to the native saddle shapes of the heart valve, even as annular dimensions change. The annuloplasty ring 1602 can be configured to preserve physiological annular movement, in some cases. In examples, the annuloplasty ring 1602 can include a dedicated suture feature/cuff. The annuloplasty ring 1602 can include markers 1606 that can be used to align the annuloplasty ring 1602 with the mitral valve 1604, such as commissure markers and a mid-posterior marker. FIG. 16B illustrates sutures/stitches 1608 attaching the annuloplasty ring 1602 to the mitral valve 1604.

FIGS. 17A-17C illustrate an example closed/complete annuloplasty ring 1702. FIG. 17A illustrates a side view of the annuloplasty ring 1702, FIG. 17B illustrates a top view of the annuloplasty ring 1702, and FIG. 17C illustrates the annuloplasty ring 1702 implanted on a mitral valve 1704. The annuloplasty ring 1702 can be configured to perform at least some remodeling of the anatomy while preserving at least some flexibility of the anatomy. The annuloplasty ring 1702 can be implemented as a semi-rigid ring. The annuloplasty ring 1702 can have a smaller posterior saddle shape in comparison to the anterior saddle shape. The annuloplasty ring 1702 can have a D-shape (e.g., for smaller sizes) and/or a more circular shape (e.g., for larger sizes). The annuloplasty ring 1702 can be configured with anterior rigidity for remodeling and/or with posterior flexibility to preserve anatomical motion. In examples, the annuloplasty ring 1702 can include a dedicated suture feature/cuff. The annuloplasty ring 1702 can include markers 1706 that can be used to align the annuloplasty ring 1702 with the mitral valve 1704, such as commissure markers and a mid-posterior marker. FIG. 17C illustrates sutures/stitches 1708 attaching the annuloplasty ring 1702 to the mitral valve 1704.

FIGS. 18A-18C illustrate another example closed/complete annuloplasty ring 1802. FIG. 18A illustrates a side view of the annuloplasty ring 1802, FIG. 18B illustrates a top view of the annuloplasty ring 1802, and FIG. 18C illustrates the annuloplasty ring 1802 implanted on a mitral valve 1804. The annuloplasty ring 1802 can be configured to perform at least some remodeling of the anatomy while preserving at least some flexibility of the anatomy. The annuloplasty ring 1802 can be implemented as a semi-rigid ring and/or can have a D-shape. The annuloplasty ring 1802 can be configured to restore a size and/or shape of a heart valve. In examples, the annuloplasty ring 1802 can include increased posterior (or anterior) flexibility in comparison to anterior (or posterior) flexibility and/or other annuloplasty rings. The annuloplasty ring 1802 can include markers 1806 that can be used to align the annuloplasty ring 1802 with the mitral valve 1804, such as commissure markers. FIG. 18C illustrates sutures/stitches 1808 attaching the annuloplasty ring 1802 to the mitral valve 1804.

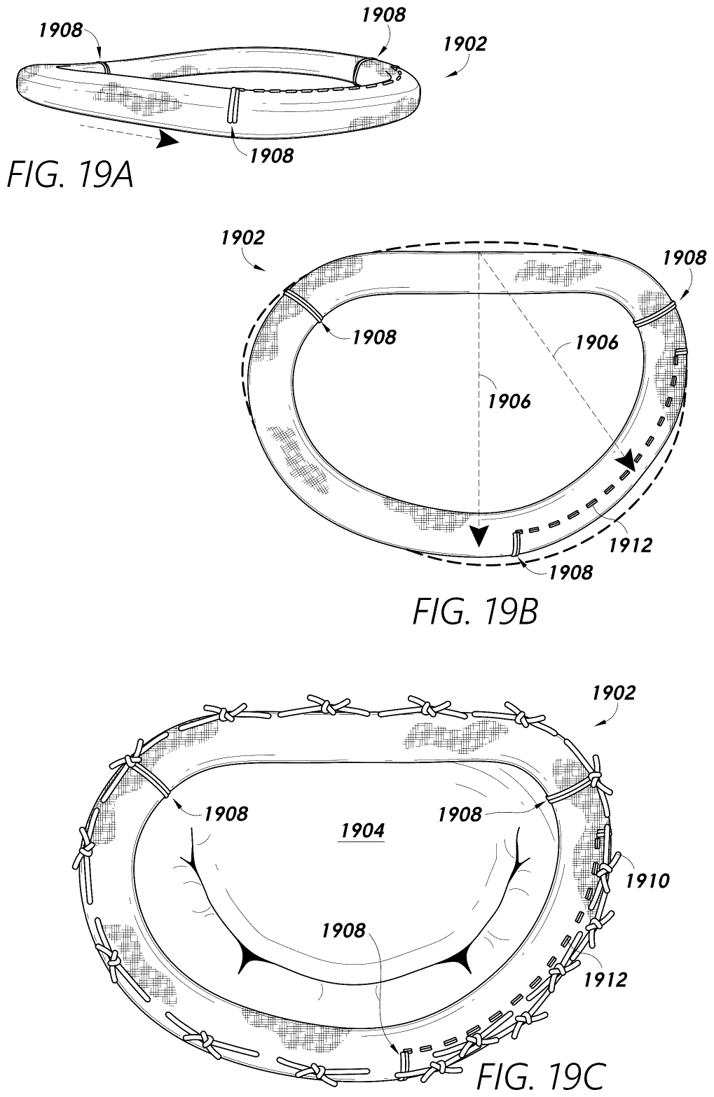

FIGS. 19A-19C illustrate another example closed/complete annuloplasty ring 1902. FIG. 19A illustrates a side view of the annuloplasty ring 1902, FIG. 19B illustrates a top view of the annuloplasty ring 1902, and FIG. 19C illustrates the annuloplasty ring 1902 implanted on a mitral valve 1904. The annuloplasty ring 1902 can be implemented as a rigid ring that is configured to perform at least some remodeling of the anatomy. The annuloplasty ring 1902 can have an asymmetrical design, as shown by lines 1906. The annuloplasty ring 1902 can have a dipped/dropped posterior segment (e.g., P3), in some cases. In examples, the annuloplasty ring 1902 can include a dedicated suture feature/cuff. The annuloplasty link ring 1902 can include markers 1908 that can be used to align the annuloplasty ring 1902 with the mitral valve 1904. FIG. 19C illustrates sutures/stitches 1910 attaching the annuloplasty ring 1902 to the mitral valve 1904. In examples, a particular segment of the annuloplasty ring 1902 can include an increased suture margin, which can be marked on the annuloplasty ring 1902 with a dashed line 1912, as shown.

### Example Flow Diagrams

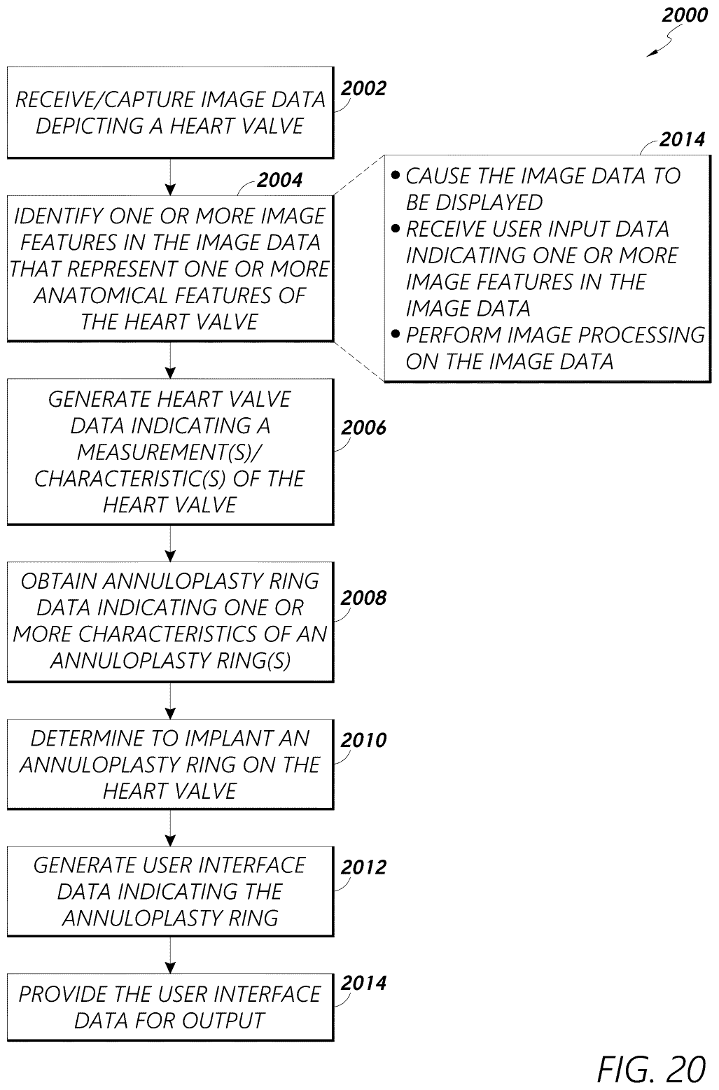

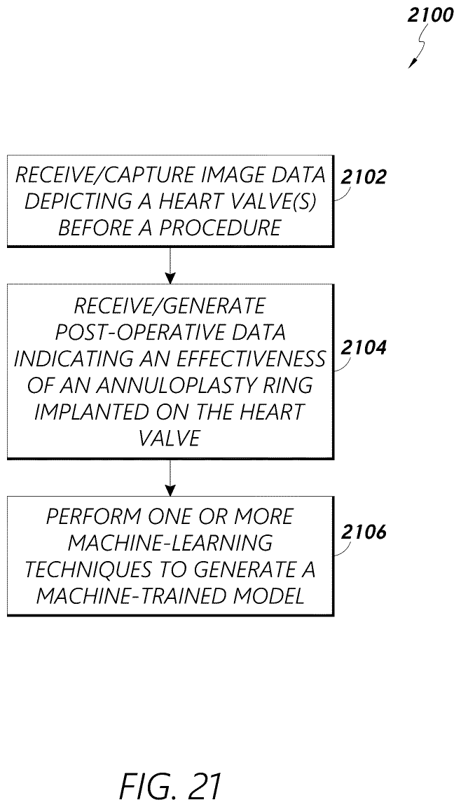

FIGS. 20 and 21 illustrate example flow diagrams of processes for performing one or more techniques discussed herein. The various blocks associated with the processes can be performed by one or more devices/systems, users, and so on. For example, one or more of the blocks can be performed by control circuitry of the service provider 906 and/or the device 902 of FIG. 9.

FIG. 20 illustrates an example process 2000 to provide a recommendation regarding an annuloplasty ring to implant for a heart valve. At block 2002, the process 2000 can include receiving/capturing image data depicting a heart valve. For example, image data of a heart valve can be captured using an imaging device, such as before or during a medical procedure. In some instances, the image data is captured with a fiduciary marker placed within a field-of-view of the imaging device, so that the image data depicts the fiduciary marker (which can indicate a predetermined distance). However, the fiduciary marker may not be used in some cases.

At block 2004, the process 2000 can include identifying one or more image features in the image data that represent one or more anatomical features of the heart valve. In some examples, an analysis can be performed automatically to identify the one or more image features, such as by performing image processing or other data processing techniques. Further, in some examples, an analysis can be performed based on user input data to identify the one or more image features. Here, the image data may be provided for display (e.g., displaying the image data, sending the image data to a display device/client device for output, etc.) and user input data can be received indicating one or more image features in the image data. The user input data can be analyzed (with or without performing image processing) to identify one or more image features in the image data that represent one or more anatomical features.

At block 2006, the process 2000 can include generating heart valve data indicating one or more measurements/characteristics of the heart valve. Such generating can be based on the one or more image features. A measurement/characteristic of a heart valve can include a surface area of an annulus, a surface area of a leaflet, a height/diameter of the annulus, a height/length/width of the leaflet, an inter-commissural distance, a distance/circumference of the annulus, an amount of coaptation gap due to improper coaptation of leaflets, a comparison/difference of the surface area of one leaflet to the surface area of the annulus, a comparison/difference of the surface area of one leaflet to another leaflet, and so on. In some examples, the heart valve data can be generated based on a predetermined distance indicated by a fiduciary marker depicted in the image data. However, the fiduciary marker may not be used in some cases, as noted above.

At block 2008, the process 2000 can include obtaining (e.g., retrieving, receiving, etc.) annuloplasty ring data indicating one or more characteristics of one or more annuloplasty rings. The annuloplasty ring data can indicate a size of an annuloplasty ring(s), a shape of the annuloplasty ring(s), a type of suture feature of the annuloplasty ring(s), a location on the annuloplasty ring(s) to suture the annuloplasty ring(s) to the heart valve(s), whether the annuloplasty ring is closed or open, a flexibility of the annuloplasty ring(s), a material of the annuloplasty ring, a structure of an inner ring body, a number of inner ring bodies, a structure of an outer covering, and so on.

At block 2010, the process 2000 can include determining to implant an annuloplasty ring on the heart valve. For example, an annuloplasty ring can be selected/identified as a recommendation for implantation at the heart valve based on heart valve data for the heart valve and/or annuloplasty ring data for one or more annuloplasty rings. In some instances, a machine-trained model can be used to select/determine the annuloplasty ring, such as from among a plurality of annuloplasty rings.

At block 2012, the process 2000 can include generating user interface data (e.g., recommendation data) indicating the annuloplasty ring. In some examples, the user interface data indicates a size of the annuloplasty ring, a shape of the annuloplasty ring, a type of suture feature of the annuloplasty ring, a location on the annuloplasty ring to suture the annuloplasty ring to the heart valve, whether the annuloplasty ring is an open or closed ring, a flexibility of the annuloplasty ring, or any other characteristic of the annuloplasty ring. The user interface data can include a recommendation to use the annuloplasty ring.

At block 2014, the process 2000 can provide the user interface data for output. For example, the user interface data can be sent to a client device/display device to display a recommendation and/or used to display the recommendation, so that a user can view information regarding a recommended type of annuloplasty ring to implant.

FIG. 21 illustrates an example process 2100 to train a model to determine an annuloplasty ring for a situation. At block 2102, the process 2100 can include receiving/capturing image data depicting a heart valve before a procedure. In some examples, the image data can be processed using image processing techniques to determine one or more characteristics/measurements of the heart valve. Further, in some examples, a physician or other user can provide user input data to indicate how/why the heart valve is not functioning properly. In some instances, any data that is received/generated prior to a procedure can be referred to as “pre-operative data.”

At block 2104, the process 2100 can include receiving/generating post-operative data indicating an effectiveness of an annuloplasty ring implanted on the heart valve. For example, following a procedure to implant the annuloplasty ring at the heart valve, image data depicting the heart valve can be captured (e.g., echocardiogram, CT scan, etc.). In some cases, the image data can be displayed to a physician or other user and the physician can rate the effectiveness of the annuloplasty ring. Additionally, or alternatively, the image data can be analyzed, such as by using image processing techniques, to determine an effectiveness of the annuloplasty ring.

At block 2106, the process 2100 can include performing one or more machine-learning techniques to generate a machine-trained model. The one or more machine-learning techniques can be based on pre-operative data, post-operative data, and/or annuloplasty ring data (which can indicate a characteristic(s) of an annuloplasty ring(s)). As such, at block 2106, a model can be trained to determine an annuloplasty ring to recommend for a situation, such as to recommend for a set of characteristics/measurements of a heart valve.

### Additional Features and Examples

1. A system comprising: control circuitry; and memory communicatively coupled to the control circuitry and storing executable instructions that, when executed by the control circuitry, cause the control circuitry to perform operations comprising: receiving image data depicting a heart valve; identifying one or more image features in the image data that represent one or more anatomical features of the heart valve; based at least in part on the one or more image features, generating heart valve data indicating a measurement of the heart valve; obtaining annuloplasty ring data indicating one or more characteristics of an annuloplasty ring; based at least in part on the heart valve data and the annuloplasty ring data, identifying the annuloplasty ring for implantation on the heart valve; and generating user interface data indicating the annuloplasty ring.

2. The system of any example herein, in particular example 1, wherein the operations further comprise: receiving additional image data depicting another heart valve before a procedure; receiving post-operative data indicating an effectiveness of another annuloplasty ring implanted on the other heart valve; and based at least in part on the additional image data and the post-operative data, performing one or more machine-learning techniques to generate a machine-trained model; wherein the identifying the annuloplasty ring includes using the machine-trained model.

3. The system of any example herein, in particular examples 1 to 2, wherein the operations further comprise: causing the image data to be displayed; and receiving user input data indicating the one or more image features in the image data; wherein the identifying the one or more image features is based at least in part on the user input data.

4. The system of any example herein, in particular examples 1 to 3, wherein the operations further comprise: performing image processing on the image data; wherein the identifying the one or more image features is based at least in part on the image processing.

5. The system of any example herein, in particular examples 1 to 4, wherein the user interface data indicates at least one of a size of the annuloplasty ring, a shape of the annuloplasty ring, whether the annuloplasty ring is a closed or open ring, or a flexibility of the annuloplasty ring.

6. The system of any example herein, in particular examples 1 to 5, wherein: the image data depicts a fiduciary marker indicating a predetermined distance; and the generating heart valve data is based at least in part on the predetermined distance.

7. The system of any example herein, in particular examples 1 to 6, wherein the measurement of the heart valve includes at least one of a surface area of a leaflet of the heart valve, a height of the leaflet, a surface area defined by an annulus of the heart valve, a height of the annulus, or an inter-commissural distance of the heart valve.

8. The system of any example herein, in particular examples 1 to 7, wherein the one or more characteristics of the annuloplasty ring comprise at least one of a size of the annuloplasty ring, a shape of the annuloplasty ring, a type of suture feature of the annuloplasty ring, whether the annuloplasty ring is a closed or open ring, or a flexibility of the annuloplasty ring.

9. A method comprising: capturing image data using an imaging device, the image data depicting a heart valve; performing, by control circuitry, image processing on the image data to identify multiple image features that represent anatomical features of the heart valve, respectively; generating, by the control circuitry, heart valve data indicative of a measurement associated with the multiple image features; retrieving, by the control circuitry, annuloplasty ring data indicative of one or more characteristics of an annuloplasty ring; determining, by the control circuitry and based at least in part on the heart valve data and the annuloplasty ring data, to use the annuloplasty ring for the heart valve; and generating, by the control circuitry, user interface data indicative of the annuloplasty ring.

10. The method of any example herein, in particular example 9, further comprising: placing a fiduciary marker within the field-of-view of the imaging device, the fiduciary marker indicating a distance; wherein the image data depicts the fiduciary marker and the heart valve data is generated is based at least in part on the distance indicated by the fiduciary marker.

11. The method of any example herein, in particular examples 9 to 10, further comprising: receiving additional image data depicting another heart valve before a procedure; receiving post-operative data indicating an effectiveness of another annuloplasty ring implanted on the other heart valve; and based at least in part on the additional image data and the post-operative data, performing one or more machine-learning techniques to generate a machine-trained model; wherein the determining to use the annuloplasty ring for the heart valve includes using the machine-trained model.

12. The method of any example herein, in particular example 11, wherein the post-operative data includes user input indicating the effectiveness of the other annuloplasty implanted on the other heart valve.

13. The method of any example herein, in particular examples 9 to 12, wherein the user interface data indicates a size of the annuloplasty ring.

14. One or more non-transitory computer-readable media storing computer-executable instructions that, when executed by control circuitry, instruct the control circuitry to perform operations comprising: receiving pre-implantation image data depicting a heart valve; identifying one or more features in the pre-implantation image data that represent one or more anatomical features of the heart valve; based at least in part on the one or more features in the pre-implantation image data, generating heart valve data indicating a measurement of the heart valve; based at least in part on the heart valve data, using a machine-trained model to determine an annuloplasty ring to implant on the heart valve; and generating recommendation data indicating the annuloplasty ring.

15. The one or more non-transitory computer-readable media of any example herein, in particular example 14, wherein the operations further comprise: receiving additional pre-implantation image data depicting another heart valve before a procedure; receiving post-operative data indicating an effectiveness of another annuloplasty ring implanted on the other heart valve; and based at least in part on the additional pre-implantation image data and the post-operative data, training a model to create the machine-trained model.

16. The one or more non-transitory computer-readable media of any example herein, in particular examples 14 to 15, wherein the operations further comprise: causing the pre-implantation image data to be displayed; and receiving user input data indicating the one or more features in the image data; wherein the identifying the one or more image features in the pre-implantation image data is based at least in part on the user input data.

17. The one or more non-transitory computer-readable media of any example herein, in particular examples 14 to 16, wherein the operations further comprise: performing image processing on the image data; wherein the identifying the one or more image features in the pre-implantation image data is based at least in part on the image processing.

18. The one or more non-transitory computer-readable media of any example herein, in particular examples 14 to 17, wherein the recommendation data indicates a size of the annuloplasty ring.

19. The one or more non-transitory computer-readable media of any example herein, in particular examples 14 to 18, wherein the measurement of the heart valve includes at least one of a surface area of a leaflet of the heart valve or a height of the leaflet.

20. The one or more non-transitory computer-readable media of any example herein, in particular examples 14 to 19, wherein the measurement of the heart valve includes at least one of a surface area defined by an annulus of the heart valve, a height of the annulus, or a distance between commissures of the heart valve.

The above description of examples of the disclosure is not intended to be exhaustive or to limit the disclosure to the precise form disclosed above. While specific examples, and examples, are described above for illustrative purposes, various equivalent modifications are possible within the scope of the disclosure, as those skilled in the relevant art will recognize. For example, while processes or blocks are presented in a given order, alternative examples can perform routines having steps, or employ systems having blocks, in a different order, and some processes or blocks may be deleted, moved, added, subdivided, combined, and/or modified. Each of these processes or blocks can be implemented in a variety of different ways. Also, while processes or blocks are at times shown as being performed in series, these processes or blocks can instead be performed in parallel or can be performed at different times.

Certain terms of location are used herein with respect to the various disclosed examples. Although certain spatially relative terms, such as “outer,” “inner,” “upper,” “lower,” “below,” “above,” “vertical,” “horizontal,” “top,” “bottom,” and similar terms are used herein to describe a spatial relationship of one device/element or anatomical structure relative to another device/element or anatomical structure, it is understood that these terms are used herein for ease of description to describe the positional relationship between element(s)/structures(s), as illustrated in the drawings. Spatially relative terms are intended to encompass different orientations of the element(s)/structures(s), in use or operation, in addition to the orientations depicted in the drawings. For example, an element/structure described as “above” another element/structure can represent a position that is below or beside such other element/structure with respect to alternate orientations of the subject patient or element/structure, and vice-versa.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

It should be understood that certain ordinal terms (e.g., “first” or “second”) can be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather can generally distinguish the element from another element having a similar or identical name (but for use of the ordinal term). In addition, as used herein, indefinite articles (“a” and “an”) can indicate “one or more” rather than “one.” Further, an operation performed “based on” a condition or event can also be performed based on one or more other conditions or events not explicitly recited. In some contexts, description of an operation or event as occurring or being performed “based on,” or “based at least in part on,” a stated event or condition can be interpreted as being triggered by or performed in response to the stated event or condition.

With respect to the various methods and processes disclosed herein, although certain orders of operations or steps are illustrated and/or described, it should be understood that the various steps and operations shown and described can be performed in any suitable or desirable temporal order. Furthermore, any of the illustrated and/or described operations or steps can be omitted from any given method or process, and the illustrated/described methods and processes can include additional operations or steps not explicitly illustrated or described.

It should be appreciated that in the above description of examples, various features are sometimes grouped together in a single example, figure, or description thereof for the purpose of streamlining the disclosure and aiding in the understanding of one or more of the various aspects of the disclosure. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the disclosure should not be limited by the particular examples described above but should be determined only by a fair reading of the claims that follow.

Unless the context clearly requires otherwise, throughout the description and the claims, the terms “comprise,” “comprising,” “have,” “having,” “include,” “including,” and the like are to be construed in an open and inclusive sense, as opposed to a closed, exclusive, or exhaustive sense; that is to say, in the sense of “including, but not limited to.”

The word “coupled”, as generally used herein, refers to two or more elements that can be physically, mechanically, and/or electrically connected or otherwise associated, whether directly or indirectly (e.g., via one or more intermediate elements, components, and/or devices. Additionally, the words “herein,” “above,” “below,” and words of similar import, when used in this application, shall refer to this application as a whole, including any disclosure incorporated by reference, and not to any particular portions of the present disclosure. Where the context permits, words in present disclosure using the singular or plural number can also include the plural or singular number, respectively.

The word “or” in reference to a list of two or more items, that word covers all of the following interpretations of the word: any of the items in the list, all of the items in the list, and any combination of the items in the list. Furthermore, as used herein, the term “and/or” used between elements (e.g., between the last two of a list of elements) means any one or more of the referenced/related elements. For example, the phrase “A, B, and/or C” means “A,” “B,” “C,” “A and B,” “A and C,” “B and C,” or “A, B, and C.”

As may be used herein, the terms “substantially” and “approximately” provide an industry-accepted tolerance for its corresponding term and/or relativity between items. For some industries, an industry-accepted tolerance is less than one percent, while for other industries, the industry-accepted tolerance can be 10 percent or more. Other examples of industry-accepted tolerances range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances can be more or less than a percentage level (e.g., dimension tolerance of less than approximately ±1%). Some relativity between items can range from a difference of less than a percentage level to a few percent. Other relativity between items can range from a difference of a few percent to magnitude of differences.

One or more examples have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks can also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

The one or more examples are used herein to illustrate one or more aspects, one or more features, one or more concepts, and/or one or more examples. A physical example of an apparatus, an article of manufacture, a machine, and/or of a process can include one or more of the aspects, features, concepts, examples, etc. described with reference to one or more of the examples discussed herein. Further, from figure to figure, the examples can incorporate the same or similarly named functions, steps, modules, etc. that can use the same, related, or unrelated reference numbers. The relevant features, elements, functions, operations, modules, etc. can be the same or similar functions or can be unrelated.

What is claimed is:

1. A system comprising:
control circuitry; and
memory communicatively coupled to the control circuitry and storing executable instructions that, when executed by the control circuitry, cause the control circuitry to perform operations comprising:
receiving image data depicting a heart valve;
identifying one or more image features in the image data that represent one or more anatomical features of the heart valve;
based at least in part on the one or more image features, generating heart valve data indicating a measurement of the heart valve;
obtaining annuloplasty ring data indicating one or more characteristics of an annuloplasty ring;
based at least in part on the heart valve data and the annuloplasty ring data, identifying the annuloplasty ring for implantation on the heart valve; and
generating user interface data indicating the annuloplasty ring.

2. The system of claim 1, wherein the operations further comprise:
receiving additional image data depicting another heart valve before a procedure;
receiving post-operative data indicating an effectiveness of another annuloplasty ring implanted on the other heart valve; and
based at least in part on the additional image data and the post-operative data, performing one or more machine-learning techniques to generate a machine-trained model;
wherein the identifying the annuloplasty ring includes using the machine-trained model.

3. The system of claim 1, wherein the operations further comprise:
causing the image data to be displayed; and
receiving user input data indicating the one or more image features in the image data;
wherein the identifying the one or more image features is based at least in part on the user input data.

4. The system of claim 1, wherein the operations further comprise:
performing image processing on the image data;
wherein the identifying the one or more image features is based at least in part on the image processing.

5. The system of claim 1, wherein the user interface data indicates at least one of a size of the annuloplasty ring, a shape of the annuloplasty ring, whether the annuloplasty ring is a closed or open ring, or a flexibility of the annuloplasty ring.

6. The system of claim 1, wherein:
the image data depicts a fiduciary marker indicating a predetermined distance; and
the generating heart valve data is based at least in part on the predetermined distance.

7. The system of claim 1, wherein the measurement of the heart valve includes at least one of a surface area of a leaflet of the heart valve, a height of the leaflet, a surface area defined by an annulus of the heart valve, a height of the annulus, or an inter-commissural distance of the heart valve.

8. The system of claim 1, wherein the one or more characteristics of the annuloplasty ring comprise at least one of a size of the annuloplasty ring, a shape of the annuloplasty ring, a type of suture feature of the annuloplasty ring, whether the annuloplasty ring is a closed or open ring, or a flexibility of the annuloplasty ring.

9. A method comprising:
capturing image data using an imaging device, the image data depicting a heart valve;
performing, by control circuitry, image processing on the image data to identify multiple image features that represent anatomical features of the heart valve, respectively;
generating, by the control circuitry, heart valve data indicative of a measurement associated with the multiple image features;
retrieving, by the control circuitry, annuloplasty ring data indicative of one or more characteristics of an annuloplasty ring;
determining, by the control circuitry and based at least in part on the heart valve data and the annuloplasty ring data, to use the annuloplasty ring for the heart valve; and
generating, by the control circuitry, user interface data indicative of the annuloplasty ring.

10. The method of claim 9, further comprising:
placing a fiduciary marker within a field-of-view of the imaging device, the fiduciary marker indicating a distance;
wherein the image data depicts the fiduciary marker and the heart valve data is generated is based at least in part on the distance indicated by the fiduciary marker.

11. The method of claim 9, further comprising:
receiving additional image data depicting another heart valve before a procedure;
receiving post-operative data indicating an effectiveness of another annuloplasty ring implanted on the other heart valve; and
based at least in part on the additional image data and the post-operative data, performing one or more machine-learning techniques to generate a machine-trained model;
wherein the determining to use the annuloplasty ring for the heart valve includes using the machine-trained model.

12. The method of claim 11, wherein the post-operative data includes user input indicating the effectiveness of the other annuloplasty implanted on the other heart valve.

13. The method of claim 9, wherein the user interface data indicates a size of the annuloplasty ring.

14. One or more non-transitory computer-readable media storing computer-executable instructions that, when executed by control circuitry, instruct the control circuitry to perform operations comprising:
receiving pre-implantation image data depicting a heart valve;
identifying one or more image features in the pre-implantation image data that represent one or more anatomical features of the heart valve;
based at least in part on the one or more image features in the pre-implantation image data, generating heart valve data indicating a measurement of the heart valve;
based at least in part on the heart valve data, using a machine-trained model to determine an annuloplasty ring to implant on the heart valve; and
generating recommendation data indicating the annuloplasty ring.

15. The one or more non-transitory computer-readable media of claim 14, wherein the operations further comprise:
receiving additional pre-implantation image data depicting another heart valve before a procedure;
receiving post-operative data indicating an effectiveness of another annuloplasty ring implanted on the other heart valve; and
based at least in part on the additional pre-implantation image data and the post-operative data, training a model to create the machine-trained model.

16. The one or more non-transitory computer-readable media of claim 14, wherein the operations further comprise:
causing the pre-implantation image data to be displayed; and
receiving user input data indicating the one or more image features in the pre-implantation image data;
wherein the identifying the one or more image features in the pre-implantation image data is based at least in part on the user input data.

17. The one or more non-transitory computer-readable media of claim 14, wherein the operations further comprise:
performing image processing on the pre-implantation image data;
wherein the identifying the one or more image features in the pre-implantation image data is based at least in part on the image processing.

18. The one or more non-transitory computer-readable media of claim 14, wherein the recommendation data indicates a size of the annuloplasty ring.

19. The one or more non-transitory computer-readable media of claim 14, wherein the measurement of the heart valve includes at least one of a surface area of a leaflet of the heart valve or a height of the leaflet.

20. The one or more non-transitory computer-readable media of claim 14, wherein the measurement of the heart valve includes at least one of a surface area defined by an annulus of the heart valve, a height of the annulus, or a distance between commissures of the heart valve.
